(19) United States
(12) Patent Application Publication
(10) Pub. No.: US 2026/0272865 A1
(43) Pub. Date: Sep. 17, 2026

(54) HYDROXYPHENYL PROPANOATE COMPOUNDS FOR TUMOUR IMMUNOTHERAPY, COMPOSITIONS AND USES THEREOF

(71) Applicant: UTI LIMITED PARTNERSHIP, Calgary (CA)

(72) Inventors: Douglas J. MAHONEY, Calgary (CA); Md Saif Uddin SIKDAR, Calgary (CA)

(21) Appl. No.: 19/167,773

(22) PCT Filed: Mar. 22, 2024
(86) PCT No.: PCT/CA2024/050352

§ 371 Date: Sep. 22, 2025

Related U.S. Application Data
(60) Provisional application No. 63/453,947, filed on Mar. 22, 2023.

(51) Int. Cl. A61K 31/192; A61K 31/196; A61K 31/216; A61K 31/4015; A61K 45/06; A61P 35/00; C07C 57/58; C07C 59/52; C07C 69/732; C07C 229/42; C07D 207/46

(52) U.S. Cl. CPC A61K 31/192; A61K 31/196; A61K 31/216; A61K 31/4015; A61K 45/06; A61P 35/00; C07C 57/58; C07C 59/52; C07C 69/732; C07C 229/42; C07D 207/46

(57) ABSTRACT

The present application relates to the use of hydroxyphenyl propanoate compounds of Formula (I), (II), (III), (IV) or (V), or pharmaceutically acceptable salts, solvates and/or ester prodrugs thereof, to increase anti-tumour immunity, to compositions comprising them, and their use, for example, in cancer immunotherapy. More particularly, the present application relates to compounds useful in the treatment of diseases, disorders, or conditions treatable by increasing anti-tumour immunity in a cell, such as cancer.

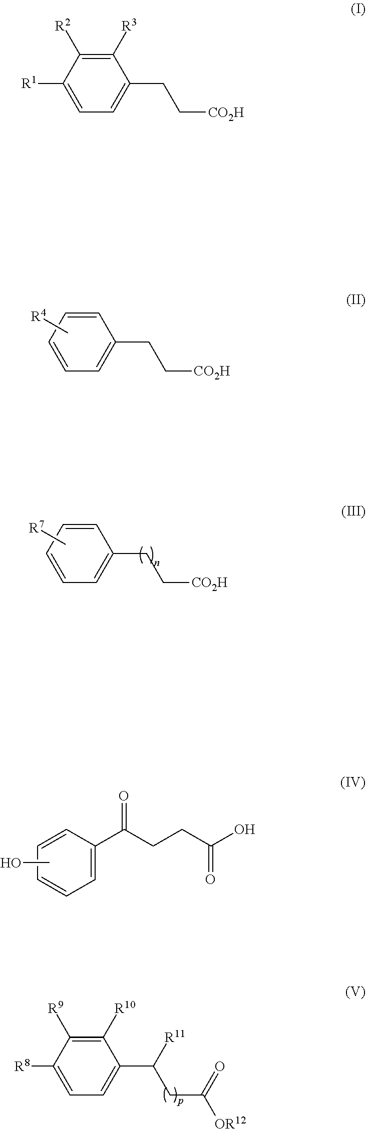

### CROSS-REFERENCE TO RELATED APPLICATIONS

The present application claims the benefit of priority from U.S. provisional patent application No. 63/453,947 filed on Mar. 22, 2023, the contents of which are incorporated herein by reference in their entirety.

### FIELD

The present application relates to hydroxyphenyl propanoate compounds that improve anti-tumour immunity, to compositions comprising them, and to their use, for example, in therapy. More particularly, the present application relates to compounds useful in the treatment of diseases, disorders, or conditions treatable by improving anti-tumour immunity, such as cancer.

### BACKGROUND

The microbiome has a significant impact on immune health and response to immune-stimulating treatments, including cancer immunotherapy. Cancer immune surveillance depends on dynamic stepwise interactions between immunogenic tumours and cells of the innate and adaptive immune system (Immunity 39, 1-10 (2013); Nature 541, 321-330 (2017)). Myeloid cells within the tumour microenvironment (TME) are at the center of this complex interplay, as they detect cancer and alarm cytotoxic T cells of its presence ((Immunity 39, 1-10 (2013); Nature 541, 321-330 (2017)). Yet myeloid cells are also commonly polarized in the TME toward immunosuppression and actively participate in cancer immune evasion (Journal for ImmunoTherapy of Cancer 7, (2019); Nat. Med. 24, (2018)). The molecular mechanisms governing myeloid cell function in the TME are being unraveled, with recent studies demonstrating an important contribution from commensal microbes and their derivative metabolites (Frontiers in Immunology 6, (2015); Cell Death Differ. 26, (2019); J. Leukoc. Biol. 100, (2016); Nature Reviews Cancer 17, (2017)). This has prompted clinical evaluation of fecal transplantation as an intervention to improve cancer outcomes and response to immunotherapy, with early results looking promising (Oncoimmunology 9, 1-8 (2020); Nat. Med. 24, (2018); Davar, D. et al. Science 371, (2021); Baruch, E N. Et al. Science 371, (2021)).

One early study showed that microbiome-derived desaminotyrosine (3,4-HPP) primes the amplification loop of type 1 interferon signalling and protects from influenza virus pathology (Science 357, 498-502 (2017)). In contrast, other studies have reported that specific isomers of the HPP metabolite can exhibit anti-inflammatory or antioxidant properties in some contexts (FASEB J. 34, 16117-16128 (2020); J. Cell. Physiol. 235, (2020); J. Agric. Food Chem. 69, (2021)). However, the molecular mechanisms by which commensal microbes influence cancer immunology remain poorly understood. Furthermore, no previous study has demonstrated the anti-tumor activity for these compounds.

### SUMMARY

The Applicants conducted deep multi-omics profiling of genetically identical mice harbouring diverse microbiomes to study the interactions between commensal microbes, their derived metabolites, and anticancer immunity. A microbiome-derived metabolite called hydroxyphenyl propanoate (HPP) has been identified that improves cancer immune surveillance, for example, by potentiating cytokine secretion into the TME through gasdermin D (GSDMD) pores on myeloid cells.

HPP molecules bind to GSDMD and can potentiate interferon regulatory factors (IRF) pathways and NF-κB pathways in a GSDMD-independent manner. The existence of multiple molecular targets in the mammalian body indicates the complex natural selection process of how a microbiome-derived metabolite can dramatically influence anti-tumour immunity. It has been shown herein that accelerated GSDMD cleavage leads to enhanced IL-1α and IL-1β release and tumour-specific CD8 T cell accumulation. Results indicate HPP compounds are useful for treating cancer/improving antitumor immunity. Accordingly, the present application includes a method of improving anti-tumour immunity in a cell, either in a biological sample or in a subject, comprising administering to the cell, an effective amount of one or more compounds of the application, wherein the compounds of the application are selected from compounds of Formula (I), or a pharmaceutically acceptable salt, solvate and/or ester prodrug thereof:

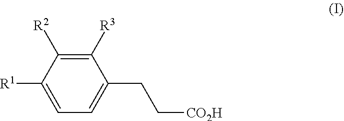

wherein
R¹, R² and R³ are, independently, OH or H, provided at least one of R¹, R² and R³ is OH.

In some embodiments, the present application includes a method of improving anti-tumour immunity in a cell, either in a biological sample or in a subject, comprising administering to the cell, an effective amount of one or more compounds of the application, wherein the compounds of the application are selected from compounds of Formula (II), (III) and (IV), or a pharmaceutically acceptable salt, solvate and/or ester prodrug thereof:

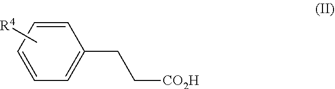

wherein
R⁴ is selected from halogen, NR⁵R⁶ and C₁₋₃alkyl; and
R⁵ and R⁶ are independently selected from H and C₁₋₃alkyl;

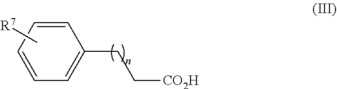

wherein
R⁷ is OH; and
n is 3-5; or

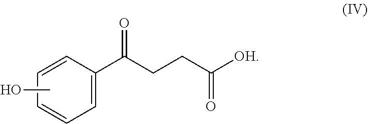

In some embodiments, the present application includes a method of improving anti-tumour immunity in a cell, either in a biological sample or in a subject, comprising administering to the cell, an effective amount of one or more compounds of the application, wherein the compounds of the application are selected from compounds of Formula (V), or a pharmaceutically acceptable salt, solvate and/or ester prodrug thereof:

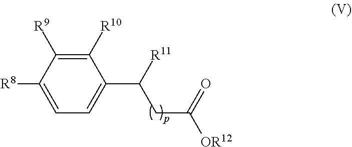

wherein
R⁸, R⁹ and R¹⁰ are, independently OH or H, provided at least one of R⁸, R⁹ and R¹⁰ is OH, or one of R⁸, R⁹ and R¹⁰ is selected from halogen, NR¹³R¹⁴ and C₁₋₃alkyl, and the other two of R⁸, R⁹ and R¹⁰ are H;
R¹³ and R¹⁴ are independently selected from H and C₁₋₃alkyl;
R¹¹ is selected from H and ═O;
R¹² is selected from H, C₁₋₄alkyl and succinimide; and
p is 1-4.

The present application also includes a method of improving anti-tumour immunity, comprising administering a therapeutically effective amount of one or more compounds of the application to a subject in need thereof.

The present application also includes a method of treating cancer in a cell in need thereof, either in a biological sample or in a subject, comprising administering an effective amount of one or more compounds of the application to the cell.

The present application also includes a method of treating cancer comprising administering a therapeutically effective amount of one or more compounds of the application to a subject in need thereof.

The present application also includes a method of treating cancer comprising administering, to a subject in need thereof, a therapeutically effective amount of one or more compounds of the application, in combination with another known agent for treating cancer or another known cancer therapy.

The present application also includes a pharmaceutical composition comprising one or more compounds of the application and a pharmaceutically acceptable carrier, wherein the one or more compounds of the application are present in the composition in an amount effective to increase anti-tumour immunity or to treat cancer.

In some embodiments, the pharmaceutical composition further comprises one or more additional anti-cancer agents.

The present application further includes a method of increasing immune surveillance by increasing GSDMD cleavage while partially protecting from cell death and potentiating the NF-κB pathway through facilitating the release of pro-inflammatory cytokines such as IL-1α and IL-1β in a cell, either in a biological sample or in a subject, comprising administering an effective amount of one or more compounds of the application to the cell.

The present application includes a method of increasing anti-tumour immunity by increasing tumour interstitial IL-1β release and tumour-specific CD8 T cell accumulation in the TME in a cell, either in a biological sample or in a subject, comprising administering an effective amount of one or more compounds of the application to the cell.

The present application also includes a method of treating a disease, disorder or condition that is treatable by increasing GSDMD cleavage, comprising administering a therapeutically effective amount of one or more compounds of the application to a subject in need thereof.

The present application also includes a method of treating a disease, disorder or condition that is treatable by increasing tumour interstitial IL-1β release and tumour-specific CD8 T cell accumulation in the TME, comprising administering a therapeutically effective amount of one or more compounds of the application to a subject in need thereof.

The present application also includes a method of treating a disease, disorder or condition that is treatable by increasing GSDMD cleavage comprising administering, to a subject in need thereof, a therapeutically effective amount of one or more compounds of the application in combination with another known agent useful for treatment of a disease, disorder or condition treatable by accelerating GSDMD cleavage.

The present application also includes a method of treating a disease, disorder or condition that is treatable by increasing tumour interstitial IL-1β release and tumour-specific CD8 T cell accumulation in the TME comprising administering, to a subject in need thereof, a therapeutically effective amount of one or more compounds of the application in combination with another known agent useful for treatment of a disease, disorder or condition treatable by increasing tumour interstitial IL-1β release and tumour-specific CD8 T cell accumulation in the TME.

In some embodiments the disease, disorder or condition that is treatable by increasing GSDMD cleavage, is cancer. In some embodiments, the disease, disorder or condition that is treatable by increasing tumour interstitial IL-1β release and tumour-specific CD8 T cell accumulation in the TME, is cancer.

The present application also includes a method of increasing the efficacy of one or more additional agents to treat cancer and/or cancer therapies comprising administering to a subject in need thereof an effective amount of one or more compounds of the application, in combination with an effective amount of the one or more additional agents to treat cancer and/or cancer therapies.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### BRIEF DESCRIPTION OF THE DRAWINGS

The present application will be described in greater detail with reference to the attached drawings and Tables in which:

FIG. 1 shows that the host microbiome is associated with its capacity for tumour immune surveillance. a-b, PcoA and relative abundance of microbiota at family level in fecal microbiome samples of CR, TAC or JAX C57BL/6 female mice before tumour implantation (n=5/group, two combined experiments, weighted UniFrac distance, pairwise PERMANOVA test); c-e, survival curves for a titration of M3-9-M tumour cells orthotopically implanted into the gastrocnemius muscle of female C57BL/6 mice sourced from CR, TAC or JAX (n=4-5/group, single experiment); f, overall survival for orthotopically implanted M3-9-M tumour into CR, TAC and JAX C57BL/6 female mice (n=10-13/group, two combined experiments, Log-Rank test); g, effect of CD8 T cell depletion using anti-CD8 (clone 2.43) neutralizing antibody on overall survival for orthotopically implanted M3-9-M into JAX C57BL/6 female mice (n=4/group, single experiment); h, flow cytometric detection of the frequency of CD8 T cells within M3-9-M tumour mass after 18 days of orthotopic implantation (n=5/group, two combined experiments, unpaired t-test); i, the qPCR cycle number of 16S gene for fecal microbial DNA of the mice receiving drinking water supplemented with or without ABX for two weeks (n=6/group, two combined experiments, unpaired t-test); j, the body weight of mice receiving drinking water supplemented with or without ABX (n=5/group, two combined experiments); k, overall survival for orthotopically implanted M3-9-M tumour into CR C57BL/6 female mice receiving normal or ABX-supplemented drinking water (n=8-12/group, two combined experiments, Log-Rank test); I, overall survival for orthotopically implanted into M3-9-M tumour into JAX C57BL/6 female mice receiving normal or ABX-supplemented drinking water (n=11-12/group, two combined experiments, Log-Rank test).

FIG. 2 shows that divergent microbiomes create differing capacities for tumour immune surveillance in genetically identical hosts. a, steps followed for the production of IMDM mice; b-d, PcoA of the fecal microbiome atthe end of each production step for IMDM mice (circular dot represent each mouse, PERMANOVA test, weighted UniFrac method); e, PcoA of the fecal microbiome of 6-8 week old IMDM-CR vs. -JAX mice, combining 3 litters over 3 generations (circular dot represent each mouse, PERMANOVA test, weighted UniFrac method); f-g, relative abundance of fecal bacteria in IMDM mice, identified through 16S amplicon sequencing; h, work flow for i-n; i-j, tumour growth kinetics and overall survival for M3-9-M tumours grown in IMDM-CR vs. -JAX female mice (n=10-12/group, two combined experiments, two-way ANOVA for growth kinetics, Log-Rank test for overall survival); k-l, tumour growth kinetics and overall survival for M3-9-M tumours grown in IMDM-CR vs. -JAX male mice (n=7/group, two combined experiments, two-way ANOVA for growth kinetics, Log-Rank test for overall survival). m-n, tumour growth kinetics and overall survival for M3-9-M^{OVA} tumours grown in IMDM-CR vs. -JAX male mice (n=12-25/group, two combined experiments, two-way ANOVA for growth kinetics, Log-Rank test for overall survival); o, flow cytometry for the detection of OVA-specific CD8 T cells within M3-9-M^{OVA} tumours grown for 18 days (n=6/group, two combined experiments, unpaired t-test).

FIG. 3 shows that divergent microbiome causes distinctive metabolomic profile within genetically identical hosts and a specific class of metabolite is associated with better host tumour immune surveillance capacity. a-b, PC plot and Spearman correlation heatmap of the fecal metabolome of IMDM-CR vs. -JAX male mice (samples pooled from two different experiments, ellipses drawn with 95% confidence interval); c-d, PC plot and Spearman correlation heatmap of the orthotopically implanted M3-9-M^{OVA} tumour metabolome of IMDM-CR vs. -JAX male mice (samples pooled from two different experiments, ellipses drawn with 95% confidence interval); e-f, PC plot and Spearman correlation heatmap of the fecal metabolome of IMDM-JAX female mice receiving normal vs. ABX-supplemented water (samples pooled from two different experiments, ellipses drawn with 95% confidence interval); g-h, PC plot and Spearman correlation heatmap of the orthotopically implanted M3-9-M tumour metabolome of IMDM-JAX female mice receiving normal or ABX-supplemented water (samples pooled from two different experiments, ellipses drawn with 95% confidence interval); i-j, volcano plots of the fecal and tumour metabolites of IMDM-CR vs. -JAX male mice, respectively (samples pooled from two different experiments, false discovery rate adjusted); k-n, relative level of the select metabolites in feces and M3-9-M^{OVA} tumour of IMDM-CR vs. -JAX male mice (samples pooled from two different experiments, unpaired t-tests). Same metabolomic datasets were used to generate different types of graphs for identical experimental groups.

FIG. 4 shows that divergent microbiomes cause distinctive metabolomic profiles within genetically identical hosts and identification of the metabolite associated with the capacity for tumour immune surveillance. a-b, PC score and Spearman correlation heatmap of the fecal metabolome of IMDM-CR vs. -JAX female mice (samples pooled from two different experiments, ellipses drawn with 95% confidence interval); c-d, PC score and Spearman correlation heatmap of the orthotopic M3-9-M RMS metabolome implanted in IMDM-CR vs. -JAX female mice (samples pooled from two different experiments, ellipses drawn with 95% confidence interval); e-f, fecal and tumour metabolites of IMDM-CR vs. -JAX female mice selected by volcano plots with fold change threshold (x) 2 and t-test threshold (y) 0.05 (samples pooled from two different experiments, FDR adjusted, log transformed p values); g-h, relative level of L-gln metabolite in feces and M3-9-M RMS tumour of IMDM-CR vs. -JAX female mice (samples pooled from two different experiments, unpaired t-test); i-j; relative level of HPP metabolite in feces and M3-9-M RMS of IMDM-CR vs. -JAX female mice (samples pooled from two different experiments, unpaired t-test); k-l, relative level of HPP metabolite in fecal and M3-9-M RMS tumour of IMDM-JAX female mice receiving normal vs. ABX-supplemented water (samples pooled from two different experiments, unpaired t-test). Same metabolomic datasets were used to generate different types of graphs for identical experimental groups.

FIG. 5 shows HPP metabolite treatment strategy determination. a, structure of exemplary HPP isomers; b, workflow for c-d; c, overall survival for different doses of intraperitoneal 3,2-HPP treatments (single experiment); d, change in body weight of the mice upon receiving different doses of intraperitoneal 3,2-HPP treatments (single experiment); e, workflow for f; f, the level of 3,2-HPP metabolite in serum at different time points after intraperitoneal delivery of 83 mg/kg bodyweight of mice (single experiment); g, workflow for h; h, change in body weight of the mice upon receiving different metabolites (single experiment); i, workflow for j-k; j-k, overall survival for orthotopically implanted B16.F10 and M3-9-M^{OVA} into IMDM-CR mice that were treated with vehicle or 3,2-HPP metabolite after 7 days of tumour implantation (two combined experiments, Log-Rank test); I, workflow for m-p; m-p, flow cytometry for measuring the effectiveness of anti-CD8 neutralizing antibody (2.43 clone; i. p.) in mice, m, vehicle, n, 3,2-HPP treatment, o, anti-CD8 antibody treatment, p, anti-CD8 antibody+3,2-HPP treatment.

FIG. 6 shows that exemplary microbiome-derived metabolite 3,2-HPP improves antitumour immunity. a, workflow for b-e; b-e, overall survival curves for immune surveillance of M3-9-M in female, M3-9-M^{OVA} in male, B16.F10 in female and M3-9-M in male IMDM-CR mice, respectively, receiving i.p. treatments of metabolites initiated from day 1 (two combined experiments, Log-Rank test); f, workflow for g-h; g-h, effects of ABX on overall survival curves for immune surveillance of M3-9-M in IMDM-JAX and IMDM-CR female mice, respectively, receiving i.p. treatments of 3,2-HPP (two combined experiments, Log-Rank test); i, workflow for j-k; j-k, flow cytometry for detection of OVA-specific CD8 T cell in the TME of M3-9-M^{OVA} in IMDM-CR male mice, 18 days post-tumour implantation, receiving 3,2-HPP treatments, and the expression of exhaustion markers on this cell type, respectively (two combined experiments, unpaired t-test); I, workflow for m; m, effects of CD8 T cell neutralization on overall survival curves for immune surveillance of M3-9-M in IMDM-CR female mice, receiving i.p. treatments of 3,2-HPP (Log-Rank test, single experiment); n, workflow for o-p; o-p, effects of anti-PD-1 and anti-PD-L1 therapies on overall survival curves for immune surveillance of M3-9-M and B16.F10 in IMDM-CR female mice, respectively, receiving i.p. treatments of 3,2-HPP (two combined experiments, Log-rank test).

FIG. 7 shows that exemplary HPP potentiates cancer immune signalling pathways in the TME: a, workflow of the animal experiment used for scRNAseq; b, immune cell types in the TME of mice identified through scRNAseq of CD45+ cells isolated from orthotopically implanted M3-9-M^{OVA} RMS; c, number of cells used for statistical analysis in scRNAseq experiment; d, number of significantly impacted genes obtained from pairwise comparisons (Wilcoxon rank sum test); ePlot of all significantly impacted genes obtained from pairwise comparisons that overlap with each other through identical or shared pathways; f, bar graph of transcriptional regulators of 3,2-HPP treatment impacted genes; g-h, NF-κB and IRF induction in mouse RAW-Dual™ cells with LPS and VSV treatment, respectively, in the presence of HPP metabolites (three combined experiments, one-way ANOVA test); i, workflow for animal experiments used in j-l; j-l, overall survival curves showing immune surveillance of M3-9-M and M3-9-M^{OVA} in male and B16.F10 in female IMDM-CR mice, respectively, receiving i.p. treatments of HPP metabolites initiated from day 1 (two combined experiments; Log-rank test).

FIG. 8 shows that exemplary 3,2-HPP metabolite treatment impacts the expression of genes in different immune cell subsets in TME. a-f, the genera plots showing overlapping genes which were statistically significantly impacted in pairwise comparison between IMDM-CR vs. JAX and IMDM-CP vs. IMDM-CR-HPP (Wilcox Rank Sum test).

FIG. 9 shows that exemplary HPP isomers (1-1, 1-2 and 1-3) potentiate NF-κB and IRF signalling pathways in myeloid cells. a, THP1-Dual™ cells treated with LPS in the presence of vehicle or HPP metabolites for NF-κB pathway induction from 0 to 24 hours (two combined experiments); b-f, THP1-Dual™ cells treated with different NF-κB pathway inducers at the presence or absence of HPP isomers for 16 hours (three combined experiments, one-way ANOVA test); g-m, THP1-Dual™ cells treated with different IRF pathway inducers at the presence or absence of HPP isomers for 16 hours (three combined experiments, one-way ANOVA test); n-o, THP1-Dual™ cells treated with LPS to induce NF-κB and VSV to induce IRF pathway, respectively, in the presence or absence of L-gln for 16 hours (three combined experiments).

FIG. 10 shows that exemplary HPP isomers (1-1, 1-2 and 1-3) potentiates NF-κB signalling and antitumour immunity by binding with GSDMD. a, NF-κB induction in human THP1-Dual™ reporter cells treated with LPS and HPP molecules for 16 hours (six combined experiments, one-way ANOVA test); b, identification of potential HPP targets in THP1-Dual™ cells by performing TPP technique (two combined experiments, proteomic coverage: 4301, NPARC test); c, protein-protein interaction network between the HPP target hits (red) and the transcriptional regulators of 3,2-HPP treatment impacted genes (yellow); d, GSDMD protein denaturation curve impacted by 3,2-HPP treatment (two-combined experiments, NPARC test); e, workflow for f-g; f-g, tumour growth kinetics and overall survival for orthotopically implanted M3-9-M^{OVA} RMS into male WT vs. GSDMD-KO mice receiving vehicle or 3,2-HPP metabolite treatments (n=8/group; two combined experiments, two-way ANOVA test for growth kinetics, Log-Rank test for overall survival); h, response of HEK-Blue™ IL-1β cell (NF-κB/AP-1 induction by IL-1 receptor signalling) to the supernatants of the THP1-WT vs. -GSDMD-KO cells treated with LPS and HPP molecules (three combined experiments, one-way ANOVA test); i, response of HEK-Blue™ IL-1β cell to the supernatants of the THP1-WT vs. -GSDMD-KO cells treated with LPS and HPP molecules followed 3 hours later by adding NG (three combined experiments, one-way ANOVA test); j, western blot of THP-1 cells treated with different conditions (representative of two experiments); k, quantification of GSDMD peptides from a publicly available proteomic profile of stage IV melanoma patients undergoing anti-PD-1 treatment (R, n=40 and NR, n=27); l-m, the supernatants of huPBMC treated with LPS and HPP molecules followed 3 hours later by adding NG were tested for: I, HEK-Blue™ IL-1β cell response (three combined experiments, one-way ANOVA test); m, level of released LDH (three combined experiments, one-way ANOVA test).

FIG. 11 shows the expression of genes involved in inflammasome pathway identified through scRNAseq.

FIG. 12 shows that exemplary 3,2-HPP metabolite treatments significantly shifts the cytokine milieu in TME. The cytokine level of the tumour interstitial fluid isolated from orthotopic M3-9-M^{OVA} tumours implanted into male JAX C57BL/6 mice after 14 days. 3,2-HPP metabolite treatments were initiated 1 day post tumour implantation. (two combined experiments; one-way ANOVA test).

FIG. 13 shows that exemplary HPP isomers (1-1, 1-2 and 1-3) accelerate immune signalling pathways by facilitating gasdermin D cleavage. a-e, THP1-WT vs. -GSDMD-KO cells were treated with LPS in the presence or absence of HPP metabolites. IgG1 isotype (clone T8E5) or anti-IL-1α (clone 7D4) and anti-IL-1β (clone 4H5) antibodies were added to neutralize secreted IL-1 cytokine when needed and the supernatants were tested for: a, NF-κB induction by THP1-Dual™ cells (three combined experiments, one-way ANOVA test); b, the response of HEK-Blue™ IL-1β cell after 4 hours for NF-κB/AP-1 induction by IL-1β signalling (three combined experiments, one-way ANOVA test); c-d, the level of secreted IL−1° and IL-1β after 16 hours (two combined experiments, one-way ANOVAtest); e, the level of LDH released after 16 hours (three combined experiments, one-way ANOVA test); f-h, the supernatants of the THP1-WT vs. -GSDMD-KO cells treated with LPS in the presence or absence of HPP metabolites followed 3 hours later by adding NG were tested for: f, the response of HEK-Blue™ IL-1β cell after 4 hours for NF-κB/AP-1 induction by IL-1β signalling (three combined experiments, one-way ANOVA test); g-h, the level of LDH released after 4 and 16 hours, respectively (three combined experiments, one-way ANOVA test); i, the response of HEK-Blue™ IFN-α/β cell (IRF induction by type I IFN signalling) to the supernatants of the THP1-WT vs. -GSDMD-KO cells treated with VSV in the presence or absence of HPP metabolites; j-k; the supernatants of the human PBMC treated with LPS in the presence or absence of HPP metabolites followed 3 hours later by adding NG were tested for the level of secreted IL-1^{α} and IL-1β, respectively (two combined experiments, one-way ANOVA test).

FIG. 14 is a generalized schematic of how the exemplary microbiome-derived metabolite HPP contributes to the anti-tumour immune response.

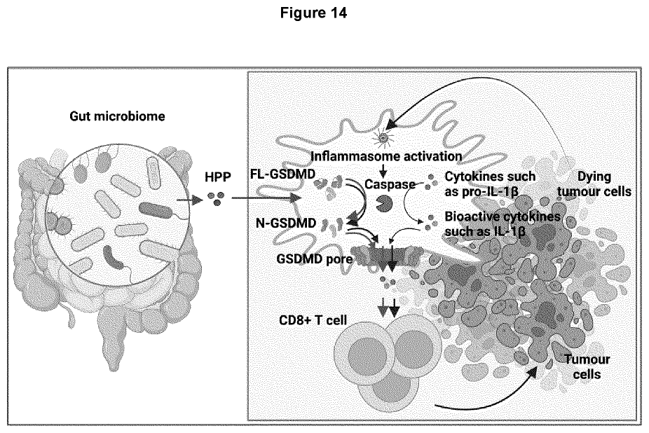

FIG. 15 shows THP1-Dua™ cells treated with LPS in the presence of vehicle or exemplary compounds II-4, II-3, II-2, II-1 and I-3 for NF-κB pathway induction for 16 hours.

FIG. 16 shows THP1-Dual™ cells treated with LPS in the presence of vehicle or exemplary compounds I-7, I-6, I-5, I-4 and I-3 for NF-κB pathway induction for 16 hours.

FIG. 17 shows THP1-Dual™ cells treated with LPS in the presence of vehicle or exemplary compounds III-2, III-1 and I-3 NF-κB pathway induction for 16 hours.

FIG. 18 shows THP1-Dual™ cells treated with LPS in the presence of vehicle or exemplary compounds IV and I-3 for NF-κB pathway induction for 16 hours.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

The term “compound of the application” or “compound of the present application” and the like as used herein refers to one or more compounds of Formula (I), (II), (III) or (IV), including pharmaceutically acceptable salts, solvates and/or ester prodrugs thereof.

The term “composition of the application” or “composition of the present application” and the like as used herein refers to a composition comprising one or more compounds the application and at least one additional ingredient.

The term “and/or” as used herein means that the listed items are present, or used, individually or in combination. In effect, this term means that “at least one of” or “one or more” of the listed items is used or present. The term “and/or” with respect to pharmaceutically acceptable salts and/or solvates thereof means that the compounds of the application exist as individual salts and hydrates, as well as a combination of, for example, a solvate of a salt of a compound of the application.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process/method steps.

As used herein, the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The present application refers to a number of chemical terms and abbreviations used by those skilled in the art. Nevertheless, definitions of selected terms are provided for clarity and consistency.

The term “cell” as used herein refers to a single cell or a plurality of cells and includes a cell either in a cell culture or in a subject.

The term “subject” as used herein includes all members of the animal kingdom including mammals. Thus, the methods and uses of the present application are applicable to both human therapy and veterinary applications.

The term “pharmaceutically acceptable” means compatible with the treatment of subjects.

The term “pharmaceutically acceptable carrier” means a non-toxic solvent, dispersant, excipient, adjuvant or other material which is mixed with an active ingredient (for example, one or more compounds of the application) to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “prodrug” as used herein means a compound, or salt and/or solvate of a compound, that, after administration, is converted into an active drug.

The term “solvate” as used herein means a compound, or a salt or prodrug of a compound, wherein molecules of a suitable solvent are incorporated in the crystal lattice.

The term “N” as used herein, for example in “4N”, refers to the unit symbol of normality to denote “eq/L”.

The term “M” as used herein, for example in 4M, refers to the unit symbol of molarity to denote “moles/L”.

The term “DMSO” as used herein refers to dimethylsulfoxide.

The term “HCl” as used herein refers to hydrochloric acid.

The term “PBS” as used herein refers to phosphate-based buffer.

The term “RT” as used herein refers to room temperature.

The term “HPLC” as used herein refers to high-performance liquid chromatography.

The term “EDTA” as used herein refers to ethylenediaminetetraacetic acid.

The term “FBS” as used herein refers to fetal bovine serum.

The term “HPP” as used herein refers to hydroxyphenyl propanoate.

The term “ICB” as used herein refers to immune checkpoint blockade.

The term “GSDMD” as used herein refers to gasdermin D.

The term “TME” as used herein refers to tumour microenvironment.

The term “PCoA” as used herein refers to principal coordinate analysis.

The term “IMDM” as used herein refers to genetically identical mice colonized with divergent complex microbiomes at birth.

The term “ABX” as used herein refers to antibiotics.

The term “UHPLC-MS” as used herein refers to ultra-high performance liquid chromatography mass spectrometry.

The term “Tregs” as used herein refers to regulatory T cells.

The term “pDc” as used herein refers to plasmacytoid dendritic cells.

The term “TPP” as used herein refers to thermal proteomic profiling.

The term “ASV” as used herein refers to amplicon sequence variants.

The term “RBC” as used herein refers to red blood cells.

The term “PVDF” as used herein refers to polyvinylidene fluoride.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of a disease, disorder or condition, stabilized (i.e. not worsening) state of a disease, disorder or condition, preventing spread of a disease, disorder or condition, delay or slowing of a disease, disorder or condition progression, amelioration or palliation of a disease, disorder or condition state, diminishment of the reoccurrence of a disease, disorder or condition, and remission (whether partial or total), whether detectable or undetectable. “Treating” and “treatment” can also mean prolonging survival as compared to expected survival if not receiving treatment. “Treating” and “treatment” as used herein also include prophylactic treatment.

“Palliating” a disease, disorder or condition means that the extent and/or undesirable clinical manifestations of the disease, disorder or condition are lessened and/or time course of the progression is slowed or lengthened, as compared to not treating the disease, disorder or condition.

The term “prevention” or “prophylaxis”, or synonym thereto, as used herein refers to a reduction in the risk or probability of a subject becoming afflicted with a disease, disorder or condition or manifesting a symptom associated with a disease, disorder or condition.

As used herein, the term “effective amount” or “therapeutically effective amount” means an amount of a compound, or one or more compounds, that is effective, at dosages and for periods of time necessary to achieve the desired result.

The expression “accelerating GSDMD cleavage” as used herein refers to promoting cleavage of the pore-forming protein gasdermin D (GSDMD) in a cell. The increased cleavage causes a therapeutic effect in the cell.

The term “increase” or “increasing” or any synonym thereof, including “improving”, “accelerating” and the like means any detectable increase in a function or amount of a targeted substance in the presence of one or more compounds of the application compared to otherwise the same conditions, except for in the absence in the one or more compounds of the application.

The term “decrease” or “decreasing” or any synonym thereof, including “lowering”, “reduction” and the like means any detectable decrease in a function or amount of a targeted substance in the presence of one or more compounds of the application compared to otherwise the same conditions, except for in the absence in the one or more compounds of the application.

The term “administered” as used herein means administration of a therapeutically effective amount of a compound, or one or more compounds, or a composition of the application to a cell or a subject.

### II. Methods and Uses of the Application

The compounds of the application have been shown to increase anti-tumour immunity and synergize with immune checkpoint blockade (ICB) therapy. HPP molecules act as broad spectrum potentiators of innate immune signalling pathways in tumour-associated myeloid cells by promoting cleavage of the pore-forming protein gasdermin D (GSDMD), an effector of canonical and non-canonical inflammasome signalling. Heightened secretion of proinflammatory cytokines from HPP-treated myeloid cells, including IL-1β, promotes NF-κB activity within tumour-infiltrating leukocytes. This leads to improved anticancer CD8 T cell function, significant tumour regression, and better long-term cancer control by immune checkpoint therapy in mice. Human peripheral blood mononuclear cells respond to HPP treatment in a similar way. GSDMD cleavage also associates with a favourable response to ICB therapy in advanced stage melanoma patients. Taken together, the present application uncovers a previously unknown mechanism of microbiome-mediated modulation of host anti-tumour immunity that is modifiable and can be harnessed to increase the efficacy of cancer immunotherapy.

Accordingly, the present application includes a method of increasing anti-tumour immunity in a cell, either in a biological sample or in a subject, comprising administering to a cell in need thereof, an effective amount of one or more compounds of the application.

In some embodiments, the compounds of the application are compounds of Formula (I), or a pharmaceutically acceptable salt, solvate and/or ester prodrug thereof:

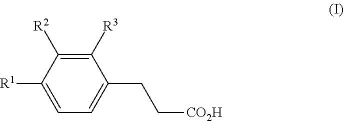

wherein
R¹, R² and R³ are, independently OH or H, provided at least one of R¹, R² and R³ is OH.

In some embodiments, the compounds of the application are selected from:

| Compound | | |
|---|---|---|
| I.D. | Structure | CHEMICAL NAME |
| I-1 | 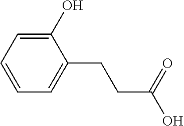 [SMILES: O=C(O)CCc1ccccc1O] | 3-(2-hydroxyphenyl)propanoic acid 3,2-HPP |
| I-2 | 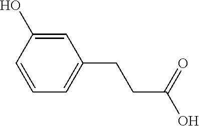 [SMILES: O=C(O)CCc1cccc(O)c1] | 3-(3-hydroxyphenyl)propanoic acid 3,3-HPP |
| I-3 | 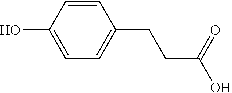 [SMILES: O=C(O)CCc1ccc(O)cc1] | 3-(4-hydroxyphenyl)propanoic acid 3,4-HPP |

or a pharmaceutically acceptable salt, solvate and/or ester prodrug thereof.

In some embodiments, the compounds ofthe application are selected from:

| Compound | | |
|---|---|---|
| I.D. | Structure | CHEMICAL NAME |
| I-4 | 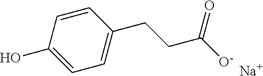 [SMILES: O=C([O-])CCc1ccc(O)cc1 ; [Na+]] | sodium 3-(4- hydroxyphenyl)propionic acid |
| I-5 | 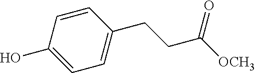 [SMILES: COC(=O)CCc1ccc(O)cc1] | methyl 3-(4- hydroxyphenyl) propionate |
| I-6 | 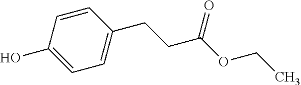 | ethyl 3-(4- hydroxyphenyl)propionate; and |
| I-7 | 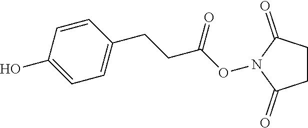 [SMILES: O=C(CCc1ccc(O)cc1)ON1C(=O)CCC1=O] | 3-(4-hydroxyphenyl)propionic acid N-hydroxysuccinimide ester. |

In some embodiments, the compounds of the application are compounds of Formula (II), (III) or (IV), or a pharmaceutically acceptable salt, solvate and/or ester prodrug thereof:

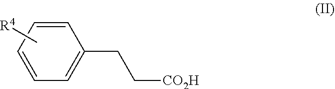

wherein
R⁴ is selected from halogen, NR⁵R⁶ and C₁₋₃alkyl; and
R⁵ and R⁶ are independently selected from H and C₁₋₃alkyl;

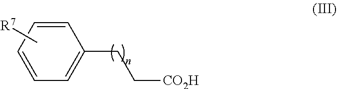

wherein
R⁷ is OH; and
n is 3-5; or

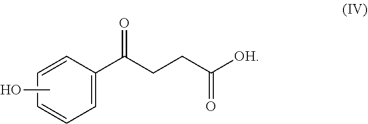

In some embodiments, the compound of Formula (II), or a pharmaceutically acceptable salt, solvate and/or ester prodrug thereof, is of the following structure:

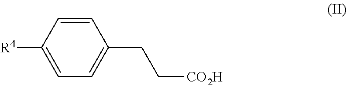

wherein R⁴ is as defined for Formula II.

In some embodiments, R⁴ is selected from F, Cl, NH₂ and CH₃.

In some embodiments, the compound of Formula (III), or a pharmaceutically acceptable salt, solvate and/or ester prodrug thereof, is of the following structure:

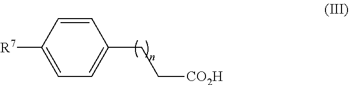

wherein R⁷ and n as defined for Formula III.

In some embodiments, the compound of Formula (IV), or a pharmaceutically acceptable salt, solvate and/or ester prodrug thereof, is of the following structure:

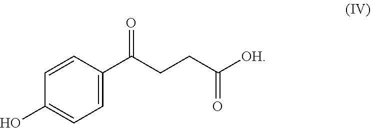 [SMILES: O=C(O)CCC(=O)c1ccc(O)cc1]

In some embodiments, the compounds of the application are selected from:

| Compound | | |
|---|---|---|
| I.D. | Structure | CHEMICAL NAME |
| II-1 | 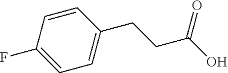 [SMILES: O=C(O)CCc1ccc(F)cc1] | 3-(4-fluorophenyl)propionic acid |
| II-2 | 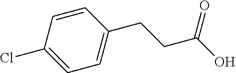 [SMILES: O=C(O)CCc1ccc(Cl)cc1] | 3-(4-chlorophenyl)propionic acid |
| II-3 | 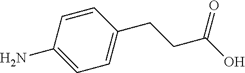 [SMILES: Nc1ccc(CCC(=O)O)cc1] | 3-(4-aminophenyl)propionic acid |
| II-4 | 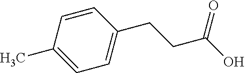 | 3-(4-methylphenyl)propionic acid |
| III-1 | 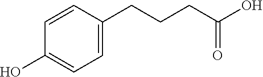 [SMILES: O=C(O)CCCc1ccc(O)cc1] | 4-(4-hydroxyphenyl)butanoic acid |
| III-2 | 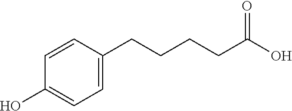 [SMILES: O=C(O)CCCCc1ccc(O)cc1] | 5-(4-hydroxyphenyl)pentatonic acid |

or a pharmaceutically acceptable salt, solvate and/or ester prodrug thereof.

In some embodiments, the compounds of the application are selected from compounds of Formula (V), or a pharmaceutically acceptable salt, solvate and/or ester prodrug thereof:

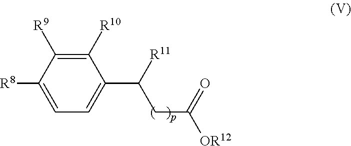

wherein
R⁸, R⁹ and R¹⁰ are, independently OH or H, provided at least one of R⁸, R⁹ and R¹⁰ is OH, or one of R⁸, R⁹ and R¹⁰ is selected from halogen, NR¹³R¹⁴ and C₁₋₃alkyl, and the other two of R⁸, R⁹ and R¹⁰ are H;
R¹³ and R¹⁴ are independently selected from H and C₁₋₃alkyl;
R¹¹ is selected from H and ═O;
R¹² is selected from H, C₁₋₄alkyl and succinimide; and
p is 1-4.

In some embodiments, R⁸, R⁹ and R¹⁰ are, independently OH or H, provided at least one of R⁸, R⁹ and R¹⁰ is OH. In some embodiments, R¹⁰ is OH and R⁸ and R⁹ are H. In some embodiments, R⁹ is OH and R⁸ and R¹⁰ are H. In some embodiments, R⁸ is OH and R⁹ and R¹⁰ are H.

In some embodiments, R⁸ is selected from halogen, NR¹³R¹⁴ and C₁₋₃alkyl, and R⁹ and R¹⁰ are H. In some embodiments, R⁸ is selected from F, Cl, NH₂ and CH₃, and R⁹ and R¹⁰ are H.

In some embodiments, R¹¹ is H. In some embodiments, R¹¹ is ═O.

In some embodiments, R¹² is selected from H, C₁₋₃alkyl and succinimide. In some embodiments, R¹² is H. In some embodiments, R¹² is CH₂—CH₃ or CH₃. In some embodiments, R¹² is succinimide.

In some embodiments, p is 1. In some embodiments, p is 2 or 3.

In some embodiments, R⁸ is OH, R⁹ and R¹⁰ are H, R¹¹ is H, R¹² is H and p is 1.

In some embodiments, R⁹ is OH, R⁸ and R¹⁰ are H, R¹¹ is H, R¹² is H and p is 1.

In some embodiments, R¹⁰ is OH, R⁸ and R⁹ are H, R¹¹ is H, R¹² is H and p is 1.

In some embodiments, R⁸ is OH, R⁹ and R¹⁰ are H, R¹¹ is H, R¹² is selected from H, CH₂—CH₃, CH₃ and succinimide and p is 1.

In some embodiments, R⁸ is selected from F, Cl, NH₂ and CH₃, R⁹ and R¹⁰ are H, R¹¹ is H, R¹² is H and p is 1.

In some embodiments, R⁸ is OH, R⁹ and R¹⁰ are H, R¹¹ is H, R¹² is H and p is 2 or 3.

In some embodiments, R⁸ is OH, R⁹ and R¹⁰ are H, R¹¹ is C(O) and p is 2.

In some embodiments the pharmaceutically acceptable salt is a base addition salt. The selection of a suitable salt may be made by a person skilled in the art (see, for example, S. M. Berge, et al., “Pharmaceutical Salts,” J. Pharm. Sci. 1977, 66, 1-19).

In some embodiments, the base addition salt suitable for, or compatible with, the treatment of subjects is any non-toxic organic or inorganic base addition salt of a compound of Formula (I). Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isooroovlamine, diethylamine, ethanolamine, trimethylamine, dicyclohexylamine, choline, and caffeine. In some embodiments, the pharmaceutically acceptable salt is a sodium salt.

Solvates of compounds of Formula (I), or a salt or ester prodrug thereof, include, for example, those made with solvents that are pharmaceutically acceptable. Examples of such solvents include water (resulting solvate is called a hydrate) and ethanol and the like. Suitable solvents are physiologically tolerable at the dosage administered.

Prodrugs of the compounds of the application, for example, conventional esters formed with the available hydroxy and/or carboxyl groups. Some common esters which have been utilized as prodrugs are phenyl esters, aliphatic (C₁-C₂₄) esters, acyloxymethyl esters, carbamates and amino acid esters. In some embodiments, the prodrug is a methyl ester, ethyl ester or N-hydroxysuccinimide ester.

The compounds of the application may further exist in varying polymorphic forms and it is contemplated that any polymorphs, or mixtures thereof, which form are included within the scope of the present application.

The present application also includes a use of one or more compounds of the application for increasing anti-tumour immunity in a cell as well as a use of one or more compounds of the application for the preparation of a medicament for increasing anti-tumour immunity in a cell. The application further includes one or more compounds of the application for use in increasing anti-tumour immunity in a cell.

As the compounds of the application have been shown to increase anti-tumour immunity, the compounds of the application are useful for treating diseases, disorders or conditions by increasing anti-tumour immunity in a cell, either in a biological sample or in a subject.

Accordingly, the present application also includes a method of treating a disease, disorder or condition that is treatable by increasing anti-tumour immunity in a cell, either in a biological sample or in a subject, comprising administering a therapeutically effective amount of one or more compounds of the application to the cell.

The present application also includes a use of one or more compounds of the application for treatment of a disease, disorder or condition that is treatable by increasing anti-tumour immunity in a cell, as well as a use of one or more compounds of the application for the preparation of a medicament for treatment of a disease, disorder or condition that is treatable by increasing anti-tumour immunity in a cell. The application further includes one or more compounds of the application for use in treating a disease, disorder or condition that is treatable by increasing anti-tumour immunity in a cell.

In some embodiments, the present application also includes a use of one or more compounds of the application for treatment of a disease, disorder or condition that is treatable by increasing GSDMD cleavage in a cell, as well as a use of one or more compounds of the application for the preparation of a medicament for treatment of a disease, disorder or condition that is treatable by increasing GSDMD cleavage in a cell. The application further includes one or more compounds of the application for use in treating a disease, disorder or condition that is treatable by increasing GSDMD cleavage in a cell.

In some embodiments, the present application also includes a use of one or more compounds of the application for treatment of a disease, disorder or condition that is treatable by increasing tumour interstitial IL-1β release and tumour-specific CD8 T cell accumulation in the TME, as well as a use of one or more compounds of the application for the preparation of a medicament for treatment of a disease, disorder or condition that is treatable by increasing tumour interstitial IL-1β release and tumour-specific CD8 T cell accumulation in the TME. The application further includes one or more compounds of the application for use in treating a disease, disorder or condition that is treatable by increasing tumour interstitial IL-1β release and tumour-specific CD8 T cell accumulation in the TME.

Compounds of the application have been demonstrated to increase anti-tumour immunity of cancer cells. In some embodiments, the disease, disorder or condition that is treatable by increasing anti-tumour immunity, is cancer.

Accordingly, the present application also includes a method of treating cancer comprising administering a therapeutically effective amount of one or more compounds of the application to a subject in need thereof. The present application also includes a use of one or more compounds of the application for treatment of cancer as well as a use of one or more compounds of the application for the preparation of a medicament for treatment of cancer. The application further includes one or more compounds of the application for use in treating cancer.

In some embodiments, the cancer is selected from, but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumour, Adult; Brain Tumour, Brain Stem Glioma, Childhood; Brain Tumour, Cerebellar Astrocytoma, Childhood; Brain Tumour, Cerebral Astrocytoma/Malignant Glioma, Childhood; Brain Tumour, Ependymoma, Childhood; Brain Tumour, Medulloblastoma, Childhood; Brain Tumour, Supratentorial Primitive Neuroectodermal Tumours, Childhood; Brain Tumour, Visual Pathway and Hypothalamic Glioma, Childhood; Brain Tumour, Childhood (Other); Breast Cancer; Breast Cancer and Pregnancy; Breast Cancer, Childhood; Breast Cancer, Male; Bronchial Adenomas/Carcinoids, Childhood; Carcinoid Tumour, Childhood; Carcinoid Tumour, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumours; Extracranial Germ Cell Tumour, Childhood; Extragonadal Germ Cell Tumour; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumour; Germ Cell Tumour, Extracranial, Childhood; Germ Cell Tumour, Extragonadal; Germ Cell Tumour, Ovarian; Gestational Trophoblastic Tumour; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumour; Ovarian Low Malignant Potential Tumour; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumours, Childhood; Pituitary Tumour; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumours; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumours, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumour, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumour. Metastases of the aforementioned cancers can also be treated in accordance with the methods described herein.

In some embodiments, the cancer is selected from one or more of solid tumours, breast cancer, colon cancer, bladder cancer, skin cancer, head and neck cancer, liver cancer, lung cancer, pancreatic cancer, ovarian cancer, prostate cancer, bone cancer, and glioblastoma. In some embodiments, the cancer is breast cancer. In some embodiments, the cancer is skin cancer. In some embodiments, the cancer is head and neck cancer. In some embodiments, the cancer is colorectal cancer (CRC). In some embodiments, the cancer is lung cancer. In some embodiments, the cancer is pancreatic cancer. In some embodiments, the cancer is ovarian cancer. In some embodiments, the cancer is prostate cancer. In some embodiments, the cancer is glioblastoma. In some embodiments, the cancer is osteosarcoma.

The present application also includes a method of treating a disease, disorder or condition that is treatable by increasing anti-tumour immunity in a cell, either in a biological sample or in a subject, comprising administering to the cell, a therapeutically effective amount of one or more compounds of the application in combination with another agent useful for treatment of a disease, disorder or condition treatable by increasing anti-tumour immunity. The present application also includes a use of one or more compounds of the application in combination with another agent useful for treatment of a disease, disorder or condition treatable by increasing anti-tumour immunity, as well as a use of one or more compounds of the application in combination with another agent useful for treatment of a disease, disorder or condition treatable by increasing anti-tumour immunity for the preparation of a medicament for treatment of a disease, disorder or condition treatable by increasing anti-tumour immunity. The application further includes one or more compounds of the application in combination with another agent useful for treatment of a disease, disorder or condition treatable by increasing anti-tumour immunity for use in treating a disease, disorder or condition treatable by increasing anti-tumour immunity surveillance.

In some embodiments, GSDMD is cleaved in the uses and methods of the application.

In an embodiment, the subject is a subject having the disease, disorder or condition.

In an embodiment, the subject is a mammal. In another embodiment, the subject is human.

In some embodiments the disease, disorder or condition that is treatable by increasing anti-tumour immunity, is cancer and the one or more compounds of the application are administered or used in combination with one or more additional agents to treat cancer and/or cancer therapies.

The present application also includes a method of increasing the efficacy of one or more additional agents to treat cancer and/or cancer therapies comprising administering to a subject in need thereof an effective amount of one or more compounds of the application, in combination with an effective amount of the one or more additional agents to treat cancer and/or cancer therapies.

The present application also includes a use of one or more compounds of the application, in combination with one or more additional agents to treat cancer and/or cancer therapies, for increasing the efficacy of the one or more additional agents to treat cancer and/or cancer therapies for treating cancer, as well as a use of one or more compounds of the application, in combination with one or more additional agents to treat cancer and/or cancer therapies, for increasing the efficacy of the one or more additional agents to treat cancer and/or cancer therapies for treating cancer. The application further includes one or more compounds of the application in combination with one or more additional agents to treat cancer and/or cancer therapies for use in increasing the efficacy of the one or more additional agents to treat cancer and/or cancer therapies for treating cancer.

In some embodiments, the one or more additional agents to treat cancer is, for example, a small molecule chemotherapy, such as cisplatin, tyrosine-kinase inhibitors, glutaminase inhibitors (e.g., glutaminase-1 (GLS1) inhibitors), and asparagine synthetase (ASNS) inhibitors. In some embodiments, the cancer therapy is, for example, radiotherapy, targeted therapy such as antibody therapy (including anti-PD-1 and/or anti-PD-L1 antibodies), immunotherapy, hormonal therapy and anti-angiogenic therapy.

In some embodiments, the immunotherapy is immune checkpoint blockade therapy. In some embodiments, the immune checkpoint blockade therapy is a PD-1 inhibitor including one or more of Pembrolizumab, Nivolumab, and Cemiplimab or a PD-L1 inhibitor including one or more of Atezolizumab, Avelumab, and Durvalumab. In some embodiments, the immune checkpoint blockage therapy is a CTLA-4 inhibitor including Ipilimumab and/or Tremelimumab. In some embodiments, the immune checkpoint blockage therapy is a LAG-3 inhibitor including Relatlimab and/or Opdualag.

In some embodiments, the chemotherapy is a chemotherapeutic agent. In some embodiments, the chemotherapeutic agent is cisplatin. Therefore, in some embodiments the disease, disorder or condition that is treatable by increasing anti-tumour immunity is cancer, and the one or more compounds of the application are administered or used in combination with cisplatin. In some embodiments, the chemotherapeutic agent is L-asparaginase (L-ASNase). Therefore, in some embodiments the disease, disorder or condition that is treatable by increasing anti-tumour immunity is cancer, and the one or more compounds of the application are administered or used in combination with L-asparaginase (L-ASNase).

In some embodiments, the small molecule therapy is a glutaminase (e.g., glutaminase-1, (GLS1)) inhibitor or an asparagine synthetase (ASNS) inhibitor. Therefore, in some embodiments the disease, disorder or condition that is treatable by increasing anti-tumour immunity, is cancer and the one or more compounds of the application are administered or used in combination with one or more glutaminase inhibitors (e.g., GLS1 inhibitors), and/or or asparagine synthetase (ASNS) inhibitors.

In some embodiments the disease, disorder or condition that is treatable by increasing anti-tumour immunity, is cancer and the one or more compounds of the application are administered or used in combination with one or more glutaminase inhibitors (e.g., GLS1 inhibitors), and/or asparagine synthetase (ASNS) inhibitors and/or L-asparaginase (L-ASNase).

When used in combination with other agents useful in treating diseases, disorders or conditions that are treatable by increasing anti-tumour immunity or cancer, it is an embodiment that the compounds of the application are administered contemporaneously with those agents. As used herein, “contemporaneous administration” of two substances to a subject means providing each of the two substances so that they are both biologically active in the individual at the same time. The exact details of the administration will depend on the pharmacokinetics of the two substances in the presence of each other and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In some embodiments, compounds of the present application are administered with another therapeutic agent simultaneously or sequentially in separate unit dosage forms or together in a single unit dosage form. Accordingly, the present application provides a single unit dosage form comprising one or more compounds of the application, an additional therapeutic agent, and a pharmaceutically acceptable carrier.

Treatment methods comprise administering to a subject a therapeutically effective amount of one or more of the compounds of the application and optionally consist of a single administration, or alternatively comprise a series of administrations, and optionally comprise concurrent administration or use of one or more other therapeutic agents. For example, in some embodiments, the compounds of the application may be administered at least once a week. In some embodiments, the compounds may be administered to the subject from about one time per two or three weeks, or about one time per week to about once daily for a given treatment. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration may be required. For example, the compounds are administered to the subject in an amount and for duration sufficient to treat the subject. In some embodiments treatment comprise prophylactic treatment. For example, a subject with early cancer can be treated to prevent progression, or alternatively a subject in remission can be treated with a compound or composition of the application to prevent recurrence.

The dosage of compounds of the application varies depending on many factors such as the pharmacodynamic properties of the compound, the mode of administration, the age, health and weiaht of the recipient, the nature and extent of the symptoms, the frequency of the treatment and the type of concurrent treatment, if any, and the clearance rate of the compound in the subject to be treated. One of skill in the art can determine the appropriate dosage based on the above factors. Compounds of the application may be administered initially in a suitable dosage that may be adjusted as required, depending on the clinical response. Dosages will generally be selected to maintain a serum level of compounds of the application from about 0.01 μg/cc to about 1000 μg/cc, or about 0.1 μg/cc to about 100 μg/cc. As a representative example, oral dosages of one or more compounds of the application will range between about 0.05 mg per day to about 1000 mg per day for an adult, suitably about 0.1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg. Compounds of the application may be administered in a single daily, weekly or monthly dose or the total daily dose may be divided into two, three or four daily doses.

In an embodiment, effective amounts vary according to factors such as the disease state, age, sex and/or weight of the subject. In a further embodiment, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art.

To be clear, in the above, the term “a compound” also includes embodiments wherein one or more compounds are referenced. Likewise, the term “compounds of the application” also includes embodiments wherein only one compound is referenced.

The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier. In embodiments of the application the pharmaceutical compositions are used in the treatment of any of the diseases, disorders or conditions described herein and the one or more compounds of the application are present in the composition in an amount effective to increase anti-tumour immunity or to treat cancer.

The compounds of the application are administered to a subject in a variety of forms depending on the selected route of administration, as will be understood by those skilled in the art. For example, a compound of the application is administered by oral, inhalation, parenteral, buccal, sublingual, nasal, rectal, vaginal, patch, pump, minipump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a compound of the application is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

In some embodiments, a compound of the application is administered parenterally. For example, solutions of a compound of the application are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, a compound of the application is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disoosal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein a compound of the application is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

In some embodiments a compound of the application is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of the application is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In some embodiments, compounds of the application may be coupled with viral, non-viral or other vectors. Viral vectors may include retrovirus, lentivirus, adenovirus, herpesvirus, poxvirus, alphavirus, vaccinia virus or adeno-associated viruses. Non-viral vectors may include nanoparticles, cationic lipids, cationic polymers, metallic nanoparticles, nanorods, liposomes, micelles, microbubbles, cell-penetrating peptides, or lipospheres. Nanoparticles may include silica, lipid, carbohydrate, or other pharmaceutically acceptable polymers.

The compounds of the application are suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

In some embodiments, the pharmaceutical compositions of the application further comprise one or more additional agents to treat cancer.

In some embodiments, the one or more additional agents to treat cancer is, for example, cisplatin, tyrosine-kinase inhibitor, glutaminase inhibitor (e.g., glutaminase-1 (GLS1) inhibitors), asparagine synthetase (ASNS) inhibitor, immune checkpoint blockade agent or antibody therapy (including anti-PD-1 and/or anti-PD-L1 antibodies).

In some embodiments, the one or more additional agents to treat cancer is, for example, immune checkpoint blockade agent. In some embodiments, the immune checkpoint blockade agent is a PD-1 inhibitor including one or more of Pembrolizumab, Nivolumab, and Cemiplimab or a PD-L1 inhibitor including one or more of Atezolizumab, Avelumab, and Durvalumab. In some embodiments, the immune checkpoint blockage agent is a CTLA-4 inhibitor including Ipilimumab and/or Tremelimumab. In some embodiments, the immune checkpoint blockage agent is a LAG-3 inhibitor including Relatlimab and/or Opdualag.

### V. Methods of Preparation of Compounds of the Application

Compounds of the present application are commercially available or can be prepared using known synthetic processes from starting materials that are also available from commercial chemical sources. The selection of a particular process to prepare a given compound of the application is within the purview of the person of skill in the art.

Salts of the compounds of the application are generally formed by dissolving the neutral compound in an inert organic solvent and adding either the desired acid or base and isolating the resulting salt by either filtration or other known means.

The formation of a desired compound salt is achieved using standard techniques. For example, the neutral compound is treated with an acid or base in a suitable solvent and the formed salt is isolated by filtration, extraction or any other suitable method.

The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Prodrugs of the compounds of the present application may be, for example, conventional esters formed with available hydroxyor carboxyl groups. For example, available hydroxy groups may be acylated using an activated acid in the presence of a base, and optionally, in inert solvent (e.g. an acid chloride in pyridine) and available carboxyl groups may be converted to an ester by activating the acid, for example by conversion to an acid chloride or using known acid coupling agents, and reacting the activated acid with a suitable nucleophilic reagent, generally in the presence of a non-nucleophilic base.

The products of the processes of the application may be isolated according to known methods, for example, the compounds may be isolated by evaporation of the solvent, by filtration, centrifugation, chromatography or other suitable method.

One skilled in the art will recognize that where a reaction step of the present application is carried out in a variety of solvents or solvent systems, said reaction step may also be carried out in a mixture of the suitable solvents or solvent systems.

### Examples

A class of microbiome-derived metabolites called hydroxyphenyl propanoate (HPP) were discovered that enhance tumour immune surveillance in mice and synergize with immune checkpoint blockade (ICB) therapy. HPP molecules act as broad spectrum potentiators of innate immune signalling pathways in tumour-associated myeloid cells by promoting cleavage of the pore-forming protein gasdermin D (GSDMD), a critical effector of canonical and non-canonical inflammasome signalling. Heightened secretion of proinflammatory cytokines from HPP-treated myeloid cells, including IL-1β, promotes NF-κB activity within tumour-infiltrating leukocytes. This leads to improved anticancer CD8 T cell function, significant tumour regression, and better long-term cancer control by immune checkpoint therapy in mice. Human peripheral blood mononuclear cells respond to HPP treatment in a similar way. GSDMD cleavage also associates with a favourable response to ICB therapy in advanced stage melanoma patients. Taken together, a previously unknown mechanism of microbiome-mediated modulation of host anti-tumour immunity was uncovered that is modifiable and can be harnessed to enhance the efficacy of cancer immunotherapy.

The following non-limiting examples are illustrative of the present application:

### Divergent Microbiomes Cause Differing Capacities for Tumour Immune Surveillance in Genetically Identical Hosts

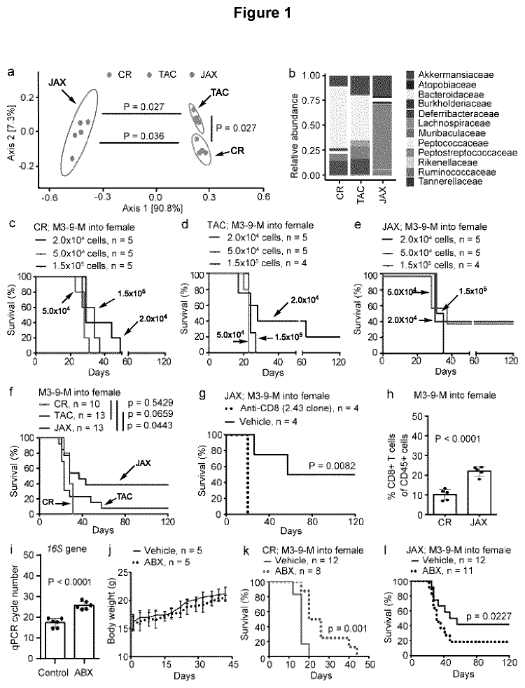

Studies have shown that inbred C57BL/6 mice obtained from different laboratories harbor divergent microbiomes and have differing capacities for cancer immune surveillance (JCI insight 3, e94952 (2018); Science 350, 1084-1089 (2015)). To confirm these observations, mice were obtained from Jackson Laboratories (JAX), Charles River Laboratories (CR), and Taconic Farms (TAC). As expected, principal coordinate analysis (PCoA) and relative abundance plots of 16S amplicon-sequenced fecal samples showed that C57BL/6 mice obtained from these different repositories were colonized with divergent microbiomes (FIG. 1a, b). Consistent with previous studies (JCI insight 3, e94952 (2018); Science 350, 1084-1089 (2015)), Muribaculaceae, previously known as Bacteroidales S24-7, was the most abundant bacterial family in the feces of mice sourced from JAX. In contrast, Bacteroidaceae was the most abundant bacterial family in mice obtained from CR and TAC. To test for impact on cancer immune surveillance, M3-9-M rhabdomyosarcoma (RMS) cells were orthotopically implanted into the gastrocnemius muscle of female mice and their tumour size monitored overtime. Tumour growth was delayed and overall survival extended in JAX mice compared to CR or TAC mice (FIG. 1c-f). This effect was dependent on CD8 T cells (FIG. 1g), which were more abundant in tumours harvested from JAX mice compared to CR mice (FIG. 1h). In a complementary approach, gut dysbiosis was induced using broad-spectrum antibiotics (ABX) at a dosing regimen that significantly reduced fecal bacterial abundance without affecting body weight (FIG. 1i,j). As expected, ABX treatment dampened M3-9-M growth in CR mice but enhanced tumour growth in JAX mice (FIG. 1k,l). These data suggest that CR and TAC mice are colonized with microbes that suppress anticancer immunity whereas JAX mice harbor a microbiome that promotes CD8 T cell activity against cancer.

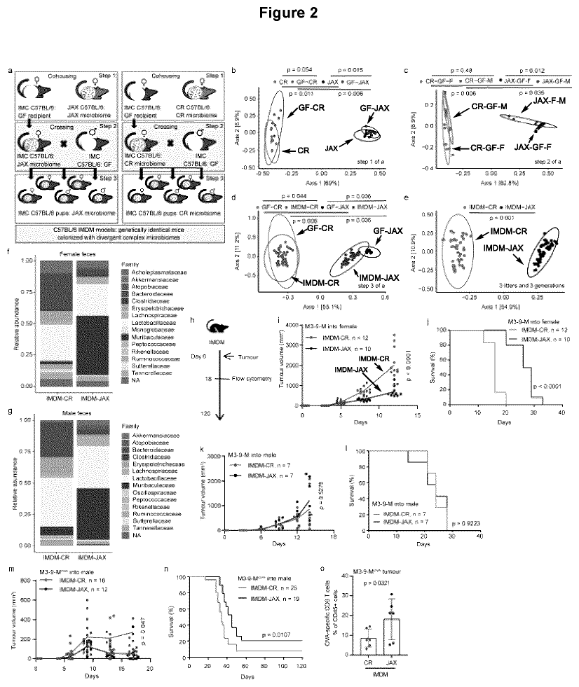

Data and previous reports using similar approaches have several important limitations, however, including that C57BL/6 mice sourced from different repositories are genetically distinct because of polymorphisms accrued over decades of inbreeding in isolation (Genome Biol. 14, 1-22 (2013); Nature 477, 289-294 (2011); Exp. Anim. 58, 141-149 (2009)) and that ABX treatment can directly influence host biology apart from microbiome depletion (Gut 64, 1732-1743 (2015)). To address these caveats, a colony of genetically-identical C57B1I/6 mice harboring JAX or CR microbiomes was developed through vertical transfer. This colony was called genetically identical mice colonized with divergent complex microbiomes at birth (IMDM) (FIG. 2a). PCoA of 16S amplicon-sequenced fecal samples showed efficient, stable and generally representative microbial transfer from donor mouse to IMDM host (FIG. 2b-e), with Muribaculaceae being the comparatively abundant bacterial family colonizing IMDM-JAX mice and Bacteroidaceae being comparatively abundant in IMDM-CR mice (FIG. 2f, g). Once established, M3-9-M cells were orthotopically implanted into female IMDM-CR and -JAX mice and monitored tumour growth over time (FIG. 2h). Consistent with the results in donor mice (FIG. 1), M3-9-M tumour growth was delayed, and overall survival extended in IMDM-JAX vs. -CR mice (FIG. 2i, j). This difference was not observed in male mice (FIG. 2k,l), in which the M3-9-M cell line is not immunogenic (Sci. Transl. Med. 6, (2014)), unless it was engineered with a model antigen (ovalbumin, OVA) (FIG. 2m,n). Together, these results provide direct support that divergent complex microbiomes create differing capacities for host tumour immune surveillance that has a significant impact on overall tumour control. Consistent with this, OVA-specific CD8 T cells were more abundant in M3-9-M^{OVA} tumours established in IMDM-JAX vs. -CR mice (FIG. 20).

Divergent Microbiomes Cause Distinctive Metabolomic Profiles within TME of Genetically Identical Hosts

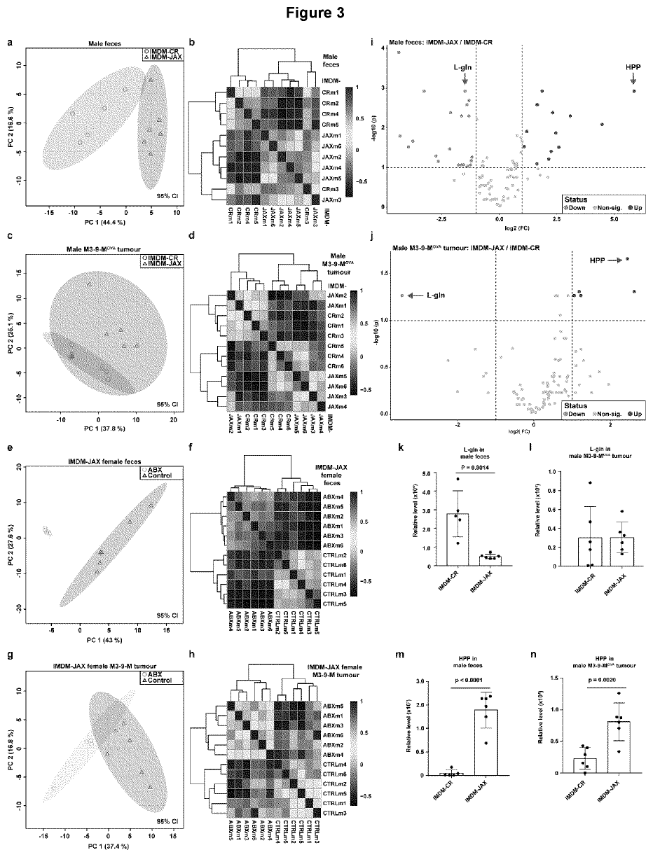

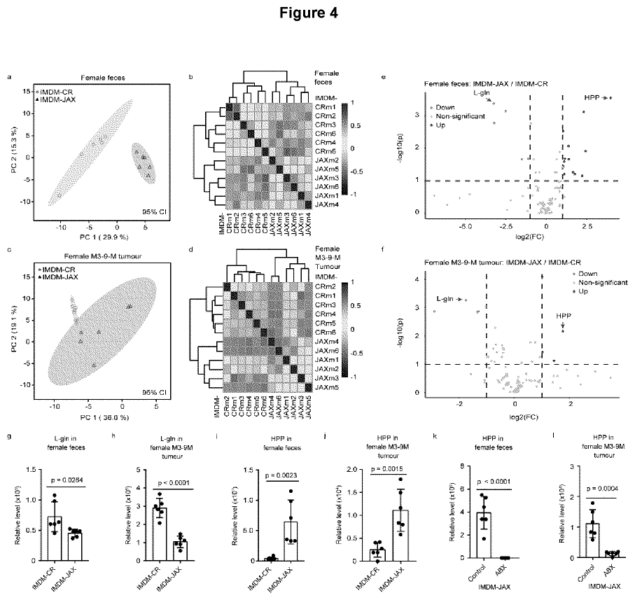

It was hypothesized that divergent compositions of microbiome engender distinctive metabolomic profiles within host tumours that modulate natural anti-tumour immunity. To test this, implanted M3-9-M and M3-9-M^{OVA} RMS tumours were extracted orthotopically from female and male IMDM mice 18 days post tumour implantation. The fecal samples were collected before tumour implantation. An untargeted ultra-high performance liquid chromatography mass spectrometry (UHPLC-MS) was conducted to detect fecal and tumour metabolites of the mice with 120 metabolite standards. Fecal and tumour metabolomes of the IMDM-CR mice clusters were found to be different than that of IMDM-JAX (FIG. 3a-d; FIG. 4a-d), suggesting that their microbiomes engender distinctive metabolomes in feces and tumour. Additionally, ABX was provided with drinking water to IMDM-JAX female mice two weeks before tumour implantation and continued throughout the experiment. Exposure to ABX altered the clustering pattern of the fecal and tumour metabolome (FIG. 3e-h), suggesting that the overall metabolome composition is modifiable.

The metabolomic datasets were further investigated to identify whether specific metabolites are associated with the capacity for tumour immune surveillance. Volcano plots of the fecal and tumour metabolome showed that some metabolites were significantly upregulated in the fecal and tumour metabolome of IMDM-JAX mice (FIG. 3i,j and FIG. 4e,f). The investigation focused on L-glutamine (L-gln) and hydroxyphenyl propanoate (HPP), because the appearance of these two metabolites were consistent across the metabolomic datasets. The relative level of L-gln was higher in fecal and tumour samples of IMDM-CR mice (FIG. 4g,h; FIG. 3k,l). On the other hand, the relative level of HPP was higher in fecal and tumour samples of IMDM-JAX mice, suggesting that the HPP metabolite is associated with better capacity for tumour immune surveillance (FIG. 3m,n; FIG. 4i,j). ABX were also found to significantly reduce the relative level of the HPP metabolite in fecal and tumour samples of IMDM-JAX mice (FIG. 4k,l), suggesting that the ABX-susceptible bacteria produce this metabolite. Next, which members of the gut microbiome produces the HPP metabolite were explored. Fecal samples of IMDM mice were split into equal half-one half for untargeted UHPLC-MS metabolomics and another half for 16S amplicon sequencing. The metabolomic and metagenomic datasets were trained by MelonnPan²⁰ tool, which is a computational method for predicting metabolite composition from microbiome sequencing data. Through this approach, 77 gut bacterial amplicon sequence variants (ASVs) were identified that are positively associated with the HPP metabolite production, including members of the Muribaculaceae family that were previously associated with enhanced anti-tumour immunity (JCI insight 3, e94952 (2018)). These results altogether establish that the divergent microbiome causes modifiable and distinctive metabolomic profiles at a distant host tumour, and that specific metabolites associate with anti-tumour immunity.

### Microbiome-Derived Metabolite HPP Enhances the Capacity for Tumour Immune Surveillance

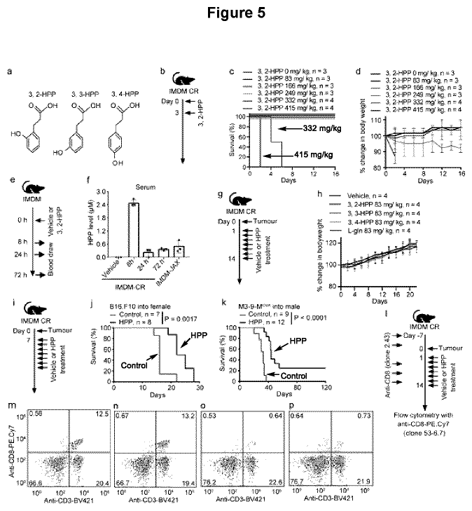

The impact of HPP treatments on tumour outcome was evaluated. Immunogenic and non-immunogenic tumours were treated with commercially available purified HPP metabolites. There are three positional isomers of HPP: 3,2-HPP, 3,3-HPP, and 3,4-HPP, which are present in animal microbiome samples, including humans. Analysis using various techniques did not detect any specific HPP isomers enriched in the biospecimens of IMDM-JAX mice, so all three HPP isomers were tested, starting with 3,2-HPP for in vivo experiments. L-gln was used as a control metabolite when needed. Endotoxin levels of the metabolite solutions used were confirmed to be within an acceptable range. Experiments showed that a dose of 83 mg/kg body weight, given every 48 hours, was suitable for treating mice without toxicity or weight loss (FIG. 5b-h).

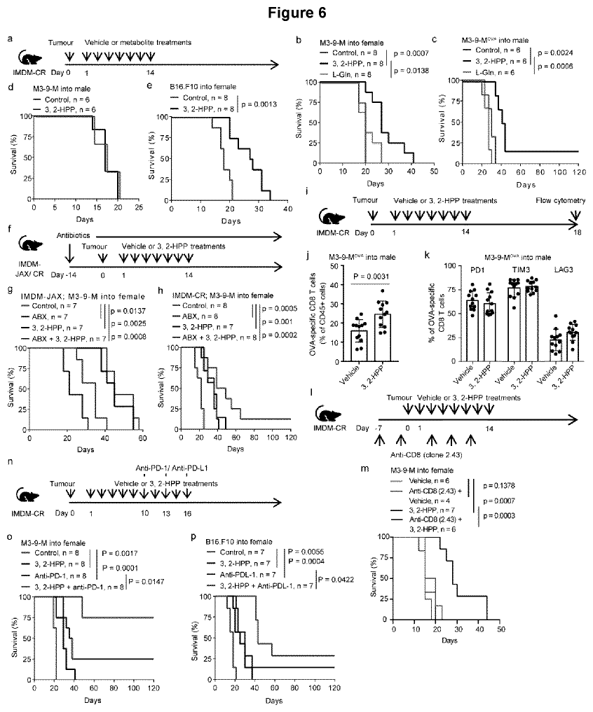

To assess the impact of 3,2-HPP on anti-tumour immunity, orthotopic immunogenic and non-immunogenic tumours were treated in IMDM-CR mice using the metabolite or a vehicle control from day 1 post-implantation (FIG. 6a). 3,2-HPP treatments were found to enhance the immune surveillance of M3-9-M tumours in female and M3-9-M^{OVA} tumours in male mice (FIG. 6b, c). Conversely, non-immunogenic M3-9-M tumours in male mice did not respond to 3,2-HPP treatments (FIG. 6d). The investigation was also extended to include subcutaneous B16.F10 melanoma tumours in female IMDM-CR mice, which have the potential to elicit an immune response due to the presence of HY-specific antigens (Cell Reports Methods 2, (2022)). 3,2-HPP treatments similarly increased the overall survival of these mice (FIG. 6e). Additionally, initiation of 3,2-HPP treatments seven days after the implantation of immunogenic tumours was still effective in IMDM-CR mice (FIG. 5i-k). These findings suggest that the 3,2-HPP metabolite has the potential to enhance anti-tumour immunity in hosts harboring an “immunosuppressive” microbiome.

Next, the impact of 3,2-HPP on anti-tumour immunity in hosts with dysbiotic microbiomes was tested. To achieve this, ABX-supplemented drinking water was administered to the mice starting two weeks prior to tumour implantation until the end of the experiment (FIG. 6f). As anticipated, ABX reduced the immune surveillance of M3-9-M tumours in female IMDM-JAX mice, while it increased it in female IMDM-CR mice (FIG. 6g, h). Interestingly, 3,2-HPP treatment increased the overall survival of IMDM-JAX mice and counteracted the negative effect of ABX (FIG. 6g). Conversely, a combination of ABX and 3,2-HPP treatment resulted in even higher overall survival in IMDM-CR mice than either treatment alone (FIG. 6h). These findings indicate that the 3,2-HPP metabolite enhances anti-tumour immunity in hosts with dysbiotic microbiomes.

It was hypothesized that treatment with the HPP metabolite enhances tumour-specific CD8 T cells in the TME, thereby improving antitumour immunity. To test this hypothesis, the effect of 3,2-HPP treatment on OVA-specific CD8 T cells within M3-9-M^{OVA} tumours in male IMDM-CR mice was evaluated (FIG. 6i). After 18 days of implantation, 3,2-HPP treatment was found to increase the frequency of OVA-specific CD8 T cells in the tumour (FIG. 6j), indicating that the tumour-specific CD8 T cell activity is linked to the therapeutic efficacy of 3,2-HPP. However, there were no significant changes in the expression of exhaustion markers such as PD1, TIM3, LAG3 on these T cells (FIG. 6k). To assess the dependence of 3,2-HPP's therapeutic outcome on CD8 T cell activity, CD8 T cells were depleted using anti-CD8 neutralizing antibody (FIG. 6l, FIG. 5l-p). Depletion of CD8 T cells abrogated the efficacy of 3,2-HPP treatment (FIG. 6m), indicating that CD8 T cell effector function is suitable for the therapeutic outcome of the metabolite.

Due to its dependence on CD8 T cell activity, the effectiveness of 3,2-HPP in combination with clinically approved ICBs, such as anti-PD-1 and anti-PD-L1 was tested. The study built upon previous reports that showed M3-9-M tumours responded to anti-PD-1 and B16.F10 tumours responded to anti-PD-L1 therapy (Sci. Transl. Med. 6, (2014); Mol. Ther. 25, (2017)). Using IMDM-CR female mice implanted with M3-9-M or B16.F10 tumours, it was found that while anti-PD-1 and anti-PD-L1 therapies alone increased overall survival in the mice, the combination of 3,2-HPP and these ICB therapies resulted in further improvement (FIG. 6n-p). This demonstrates the potential of 3,2-HPP to enhance the efficacy of ICB therapies.

The aforementioned findings demonstrate that the microbiome-derived HPP metabolite enhances the host's capacity for tumour immune surveillance. The combination of this metabolite with other cancer therapies holds potential as a promising strategy for enhancing treatment outcomes.

### HPP Enhances Anti-Tumour Immunity by Potentiating Cancer Immune Signalling Pathways in the TME

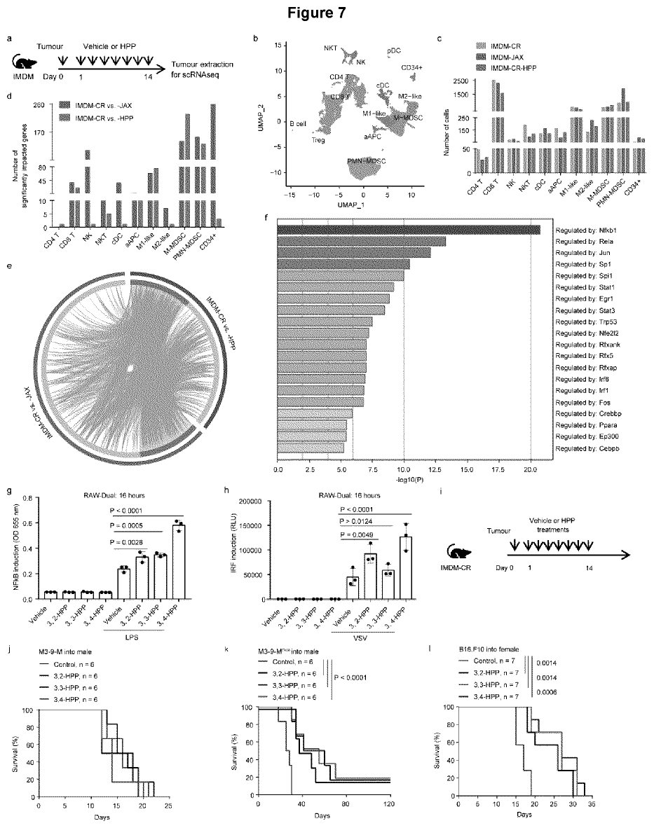

It was hypothesized that HPP metabolites enhance anti-tumour immunity by modulating cancer immune signalling pathways in the TME. To test this, single-cell RNA sequencing (scRNAseq) was used to analyze gene expression within immune cells isolated from M3-9-M^{OVA} RMS tumours grown in male IMDM-JAX, IMDM-CR and IMDM-CR mice receiving 3,2-HPP treatment (IMDM-CR-HPP). 14 distinct immune cell clusters were identified, including B cells, regulatory T cells (Tregs), plasmacytoid dendritic cells (pDC), CD8 T cells, CD4 T cells, natural killer (NK) cells, NKT cells, CD34+ cells, classical dendritic cells (cDC), atypical antigen-presenting cells (aAPC), M1-like and M2-like macrophages, monocytic myeloid-derived suppressor cells (M-MDSC), and polymorphonuclear MDSCs (FIG. 7a-c).

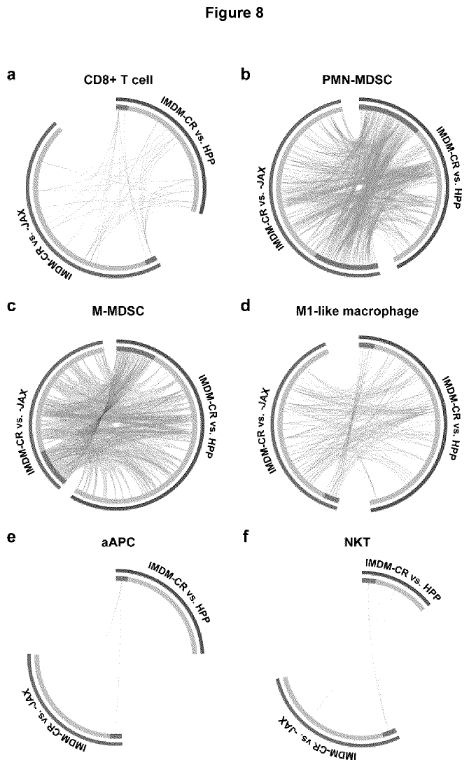

Wilcoxon rank-sum tests were performed to compare gene expression between IMDM-CR and IMDM-JAX, as well as IMDM-CR and IMDM-CR-HPP. Findings indicated that the 3,2-HPP treatment had similar effects on the number of gene expression as the JAX microbiome in multiple immune cell types (FIG. 7d). Circos plots for CD8 T cells, PMN-MDSC, M-MDSC, M1-like macrophages, aAPC, and NKT cells suggest that many of the genes affected by 3,2-HPP treatment are also involved in the same pathways as those impacted by the JAX microbiome (FIG. 8). A combined circos plot (FIG. 7e) further highlights the similarities between the impact of 3,2-HPP treatment and the JAX microbiome. Using the Metascape TRRUST database, transcriptional regulators affecting the expression of 3,2-HPP impacted genes were predicted. Several key regulators involved in cancer immunity pathways, such as NF-κB and IRF were found (FIG. 7f).

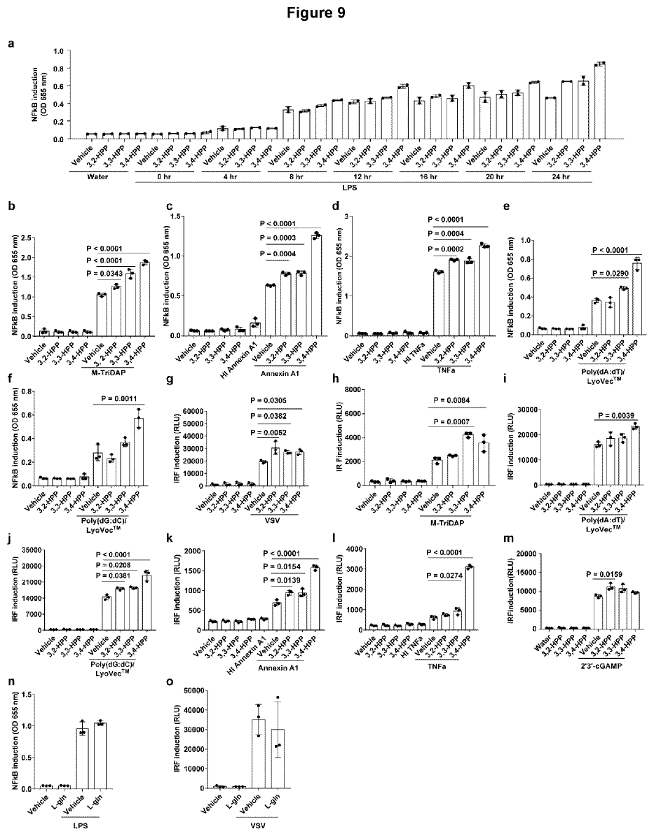

The impact of all three HPP metabolites on the activation of two cancer immune signaling pathways, NF-κB and IRF, in murine macrophage-like RAW-Dual™ reporter cells and human monocytic THP1-Dual™ reporter cells was validated. Results showed that HPP metabolites alone did not activate NF-κB and IRF signaling in either cell type. However, when combined with PRR agonists such as lipopolysaccharide (LPS), vesicular stomatitis virus (VSV) or others, HPP metabolites significantly enhanced the activation of these pathways (FIG. 7g; FIG. 9a-m). In contrast, another metabolite, L-gin, had no effect on NF-κB or IRF pathways (FIG. 9n, o). These findings suggest that HPP metabolites act as broad accelerators of NF-κB and IRF signalling when these pathways are initiated by an immune response.

Because variable effects of HPP metabolites on NF-κB and IRF pathways in cell culture were observed, their effect on tumour immune surveillance in mice was tested. After administering HPP treatment regimens one day post orthotopic tumour implantation in IMDM-CR mice, non-immunogenic M3-9-M were found not to respond to any HPP metabolites (FIG. 7j). All three HPP metabolites, however, enhanced immune surveillance of M3-9-M^{OVA} RMS and B16.F10 melanoma (FIG. 7k, I). Taken together, these results indicate that HPP metabolites enhance anti-tumour immunity through potentiating cancer immune signalling pathways that are initiated by natural tumour immune surveillance.

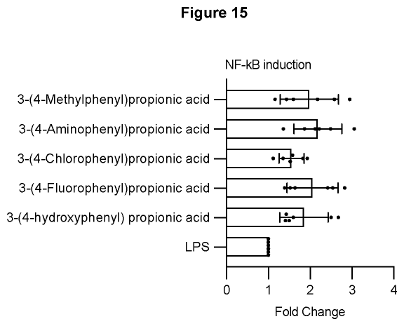

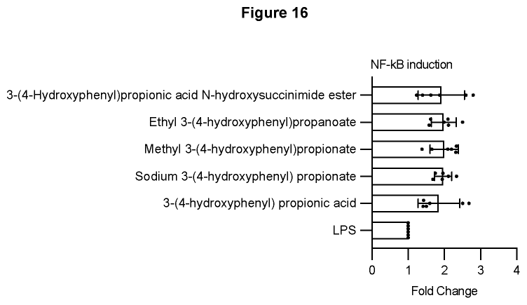

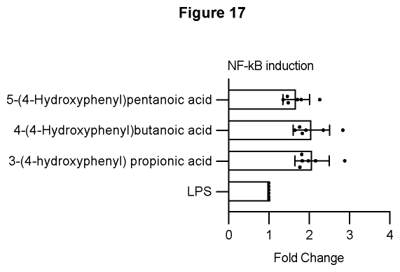

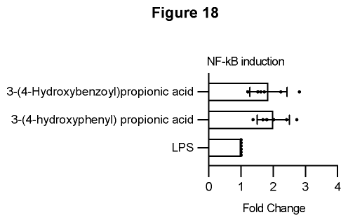

The impact of analogs of the HPP metabolites of the application on the activation ofthe NF-κB pathway in human monocytic THP1-Dual™ cells was also validated. When treated with LPS, exemplary compounds II-1, II-2, II-3, II-4, I-4, I-5, I-6, I-7, III-1, III-2 and IV significantly enhanced the activation of the NF-κB pathway (FIGS. 15-18). These findings further support the finding that HPP metabolites and analogs thereof act as broad accelerators of NF-κB signalling when this pathway is initiated by an immune response.

HPP Potentiates NF-κB Signalling and Antitumour Immunity by Binding with GSDMD

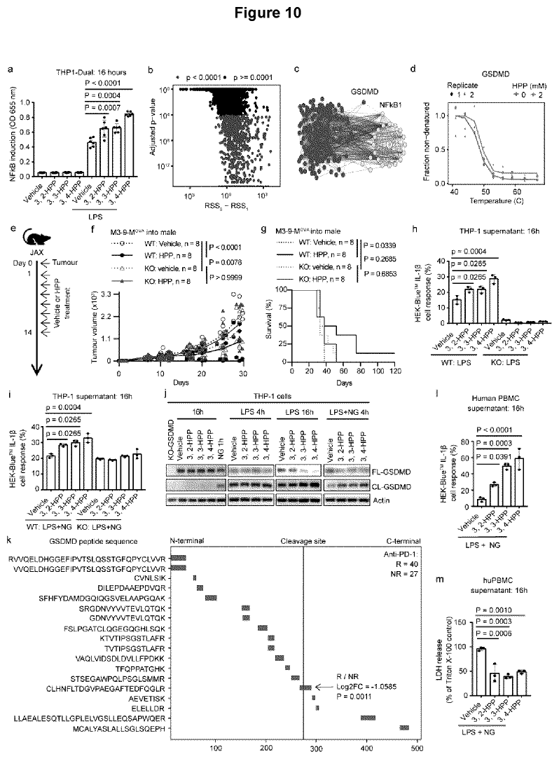

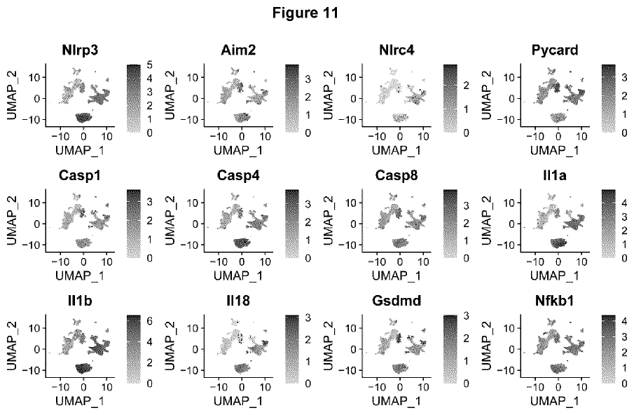

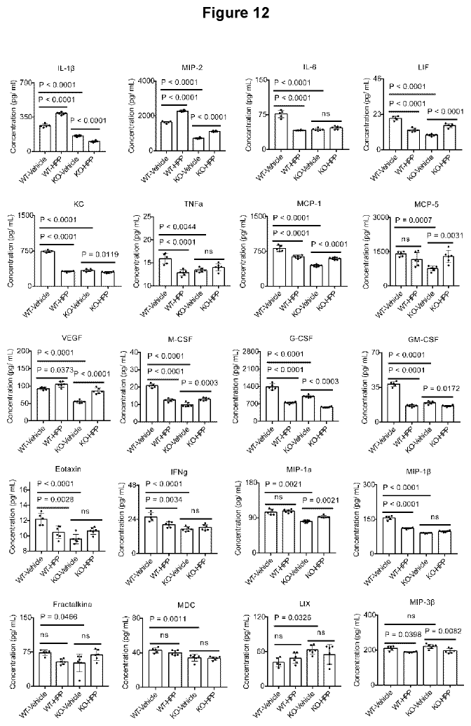

It was hypothesized that the HPP metabolite enhances cancer immunity by targeting a critical signalling protein involved in the NF-κB pathway. Since it was found that HPP enhanced NF-κB induction in human THP1-Dual™ cells when triggered by LPS (FIG. 10a), thermal proteomic profiling (TPP) was performed to determine the molecular targets of the metabolite. Through nonparametric analysis of the response curves (NPARC) (Mol. Cell. Proteomics 18, (2019)), 647 potential protein targets of 3,2-HPP metabolite were identified (FIG. 10b). To narrow down the investigation, GSDMD was focused on (FIG. 10c, d), which is a cell membrane pore-forming protein that plays a critical role in inflammasome pathway, IL-1β release and tumour immunity (Cell Rep. 34, (2021); Cell Res. 25, 1285-98 (2015); J. Immunother. Cancer 10, (2022); Cell Rep. 41, (2022); Int. Immunopharmacol. 74, (2019); J. Dig. Dis. 19, (2018); Int. J. Biol. Sci. 17, (2021); Nat. Commun. 13, 1-20 (2022); BMC Cancer 20, (2020); Oncogene 41, 5092-5106 (2022)). A correlation between the expression of inflammasome genes, GSDMD and NF-κB1 in tumour infiltrated immune cells was observed (FIG. 11). Whether 3,2-HPP targets GSDMD-mediated anti-tumour immunity was validated by orthotopically implanting M3-9-M^{OVA} RMS into the gastrocnemius muscle of male C57BL/6NJ (WT) and C57BL/6N-Gsdmdem4Fcw/J (KO) mice (FIG. 10e). Treatment with 3,2-HPP decreased tumour growth and increased overall survival in WT mice but had no effect on tumours implanted in KO mice (FIG. 10f, g). It was also found that 3,2-HPP treatment impacted the secretion of several GSDMD regulated cytokines, including a notable increase in IL-1β release (FIG. 12).

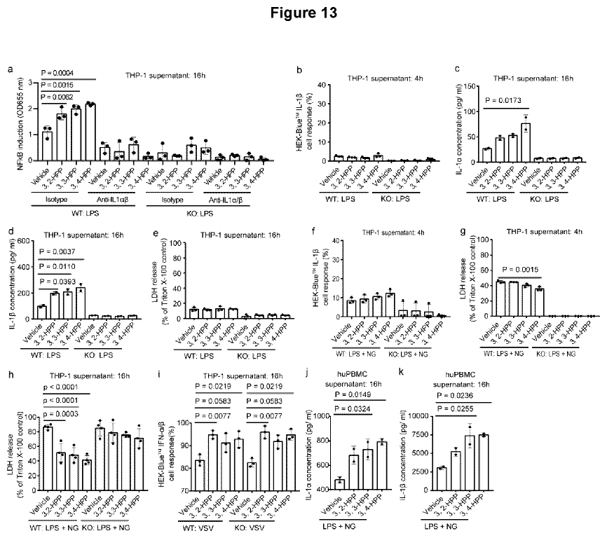

It was hypothesized that HPP-GSDMD interaction helps in facilitating specific cytokine secretion, such as IL-1β, which then triggers NF-κB induction through IL-1 receptor signaling. To test this, wild-type (WT) and GSDMD-knockout (KO) THP1 cells were treated with LPS in the presence of HPP molecules and IL-1 neutralizing antibodies. The supernatants were collected from the KO and WT cells after 16 hours, treated with IL-1 neutralizing antibodies, were found to inhibit NF-κB induction in THP1-Dual™ reporter cells (FIG. 13a). In addition, the effect of the supernatants from WT and KO THP1 cells treated with LPS were evaluated in the presence of vehicle or HPP molecules on IL-1 receptor signaling using HEK-Blue™ IL-1β cells. The supernatants collected after 16 hours from the WT cells were treated in the presence of HPP molecules enhanced IL-1 receptor signaling in HEK-Blue™ IL-1β cells (FIG. 13b; FIG. 10h). Further examination of the supernatants revealed that the presence of HPP molecules increased the release of IL-1α and IL-1β from the WT cells (FIG. 13c-d). The assessment of lactate dehydrogenase (LDH) release as a measure of cell death showed no significant changes in response to LPS or HPP treatment alone or in combination (FIG. 13e).

In another complementary approach, the HPP-GSDMD interaction in THP1 cells was examined using LPS and nigericin (NG) stimulation. Results showed that early IL-1 secretion was dependent on GSDMD activity, as HEK-Blue™ IL-1β cells responded to the early supernatants from LPS+NG-treated WT cells, but not to supernatants from GSDMD-KO cells (FIG. 13f). However, prolonged LPS+NG-induced IL-1 secretion was independent of GSDMD activity, as late supernatants from both WT and KO cells produced similar response (FIG. 10i). HPP treatments enhanced IL-1 release from WT cells while protecting from LDH release in this context (FIG. 10i; FIG. 13g, h). Additionally, HPP isomers alone did not cause GSDMD cleavage but enhanced the cleavage in the presence of LPS or LPS+NG stimulation (FIG. 10j). These results indicate that HPP molecules enhance GSDMD activity in a unique way that facilitates the release of specific NF-κB inducing cytokines while protecting from cell death. The effect of HPP-GSDMD interaction on IRF pathway induction was also evaluated. WT and KO cells were treated with VSV in the presence of HPP molecules and supernatants were collected after 16 hours to assess IRF induction through IFN-α/β receptor signalling using HEK-Blue™ IFN-α/β cells. The supernatants of both HPP-treated WT and KO cells enhanced the response of HEK-Blue™ IFN-α/β cells (FIG. 13i), suggesting that HPP facilitates this pathway in a GSDMD-independent route.

The clinical relevance of GSDMD cleavage was tested by peptide quantification from a publicly available proteomic profile of stage IV melanoma patients undergoing either tumour-infiltrating lymphocyte (TIL)-based or anti-PD-1 therapy (Cell 179, (2019)). Although data obtained from the TIL cohort was not useful for assessing the protein cleavage, an uncleaved peptide declined in responders of anti-PD-1 treatment. Caspases cleave the human GSDMD protein at residue D275 after the FLTD motif, resulting in the membrane localization of the N-terminal domain and pore formation (Nature 526, (2015); Proc. Natl. Acad. Sci. 115, (2018)). A decline of uncleaved peptide in responders therefore suggests that enhanced GSDMD cleavage is associated with a favourable response to anti-PD-1 treatment (FIG. 10k). To translate the effects of HPP metabolite to human primary peripheral blood mononuclear cells (huPBMCs), huPBMCs were treated with LPS followed by NG in the presence of HPP molecules for 16 hours and collected supernatants for cytokine assessment. HPP molecules enhanced the release of IL-1α and IL-1β (FIG. 13j, k). Moreover, the supernatant of HPP treated huPBMCs enhanced the response of HEK-Blue™ IL-1β reporter cell (FIG. 10k) and protected huPBMCs from LDH release (FIG. 10m). Taken together, the results suggest that HPP metabolites have a unique effect on GSDMD activity in myeloid cells. They enhance the release of certain cytokines that activate the NF-κB pathway, leading to improved anti-tumour immunity (FIG. 10).

### Discussion

In this study, the impact of complex microbiomes on tumour immune surveillance in genetically identical hosts was investigated. To do so, an in-house developed inbred mouse model called IMDM, which mimics the natural course of complex microbiome colonization and stability in genetically identical hosts was used. This allowed for the establishment of the causal effects of microbiomes. The findings suggest that colonization with divergent complex microbiomes can engender distinct metabolomic profiles in genetically identical hosts, leading to different capacities for tumour immune surveillance. Different microbiome-derived metabolites have been associated with improving anti-tumour immune response and immunotherapies (Science 369, 1481-1489 (2020); Nat. Metab. 2, (2020); Cell Metab. 34, (2022); Cancer Discov. 12, (2022); Sci. Immunol. 7, (2022); Sci. Immunol. 8, (2023); Cell 184, (2021)). For example, it has been shown that specific microbiome-derived metabolites trigger or potentiate type 1 interferon pathway in mononuclear phagocytes, resulting in improved anti-tumour immune response (Sci. Immunol. 7, (2022); Sci. Immunol. 8, (2023); Cell 184, (2021)). It was demonstrated that the microbiome-derived HPP metabolite can be combined with ICBs to improve treatment outcomes in hosts harbouring divergent microbiomes, including dysbiosis. Moreover, it was showed that HPP broadly potentiates innate immune signalling, such as NF-κB and IRF pathways in myeloid cells and enhance CD8 T cell dependent tumour clearance.

Like inflammation, GSDMD protein probably acts as a double-edged sword in tumour immunity. While some reports have shown that GSDMD restricts anti-tumour immunity during ICB therapy (J. Immunother. Cancer 10, (2022); Cell Rep. 41, (2022)), others argue that its activation can improve antitumour immunity and effector CD8 T cell response (Int. Immunopharmacol. 74, (2019); J. Dig. Dis. 19, (2018); Int. J. Biol. Sci. 17, (2021); Nat. Commun. 13, 1-20 (2022); BMC Cancer 20, (2020); Oncogene 41, 5092-5106 (2022)). Different molecular functions have been linked to GSDMD protein, including pro-inflammatory cytokine release with or without causing cell death (Nature 526, (2015); Immunity 48, (2018)), mucin secretion from intestinal goblet cells (Sci. Immunol. 7, (2022)), and nuclear translocation that interferes with the DNA-damage repair ability of PARP-133. Enhanced GSDMD cleavage was found to be associated with a favourable response to ICB therapy in a cohort of advanced stage melanoma patients. Additionally, HPP molecules were shown to accelerate GSDMD cleavage while partially protecting from cell death and potentiate NF-κB pathway through facilitating the release of pro-inflammatory cytokines such as IL-1α and IL-1p. IL-1β has been shown to enhance CD8 T-cell expansion, function, and anti-tumour immunity (Sci. Immunol. 6, (2021); J. Exp. Med. 210, 491-502 (2013)). HPP metabolites were also found to enhance tumour interstitial IL-1β release and tumour-specific CD8 T cell accumulation in the TME, which improves anti-tumour immunity. To conclude, it has been demonstrated that the microbiome-derived HPP metabolites boosts the capacity of host tumour immune surveillance and could be used as a treatment adjuvant in different settings. Moreover, some of these findings were validated in human cells.

### Materials and Methods

### Cell Culture

M3-9-M cells were provided by Crystal MacKall (Stanford, CA, USA). M3-9-M^{OVA} cells were generated by transfecting M3-9-M cells with the super piggyBac transposase expression vector (System Biosciences; Palo Alto, CA, USA). The American Type Culture Collection (ATCC; Manassas, VA, USA) supplied B16.F10 (CRL-6475) and THP-1 (TIB-202™) cells. InvivoGen (San Diego, CA, USA) supplied RAW-Dual™ cells, THP1-Dual™ cells, HEK-Blue™ IL-1β cells, THP1-Null2 Cells and THP1-KO-GSDMD cells. M3-9-M and M3-9-M^{OVA} cells were propagated in RPMI 1640 (Life Technologies, Carlsbad, CA, USA) supplemented with 10% heat-inactivated fetal bovine serum (FBS; Life Technologies) and 50 μM 2-mercaptoethanol (Gibco™). To maintain transgenic M3-9-M^{OVA} cells, 1 μg/mL puromycin dihydrochloride (Gibco™) was added to the culture media every other cell culture passage interval. B16.F10 (CRL-6475) cells were propagated in DMEM (Life Technologies) with 10% heat-inactivated FBS (Life Technologies).

RAW-Dual™ cells and HEK-Blue™ IL-1β cells were propagated in DMEM (Life Technologies), 4.5 g/l glucose, 2 mM L-glutamine, 10% heat-inactivated FBS, 100 μg/ml Normocin™, and Penicillin-Streptomycin (100 U/mL-100 μg/mL). To maintain the transgenic cell lines, 200 μg/mL and 100 μg/mL of Zeocin™ was added to the growth medium every other passage. THP-1 (TIB-202™), THP1-Null2 Cells, THP1-Dual™ and THP1-KO-GSDMD cells were propagated in RPMI 1640, 2 mM L-glutamine, 25 mM HEPES, 10% heat-inactivated FBS, 100 μg/mL Normocin™, and Pen-Strep (100 U/ml-100 μg/mL). To maintain THP1-Dual™ cells, 10 μg/mL blasticidin and 100 μg/mL Zeocin™ was added to the growth medium every other passage. To maintain THP1-KO-GSDMD and THP1-HMGB1-Lucia™ cells, 100 μg/mL Zeocin™ was added to the growth medium every other passage. Batches of frozen stocks for each cell line were prepared. Cells were stored by resuspending them in growth medium with 20% FBS and 10% DMSO. All cell lines were screened for mycoplasma every 8-12 weeks using the PCR Mycoplasma detection kit from Thermo Scientific.

### Animal Models

All animal experiments were performed following Canadian Council for Animal Care guidelines and University of Calgary (Canada) Health Sciences Animal Care Committee-approved protocols (AC20-0208 and AC20-0146). Animal experiments were conducted with complex microbiomes in the University of Calgary's biohazard facility. SPF C57BL/6J, C57BL/6NCr and C57BL/6NTac mice were obtained from three different commercial vendors: Jackson laboratories (breeding room number RB08), Charles River Laboratory Inc. (breeding room C62) and Taconic Farm (breeding room IBU1501C). International Microbiome Centre (IMC; University of Calgary) supplied GF mice to produce genetically identical mice colonized with divergent complex microbiomes at birth (IMDM). IMDM models were produced through cohousing and breeding in isolated cages inside the biohazard facility. Aseptic techniques were followed to avoid cross-contaminating the microbiome. Microbiome consistency was confirmed across experiments using 16S amplicon sequencing of fecal samples. Age- and sex-matched mice were randomly selected for different experimental groups. GSDMD knockout (KO) and wild-type control (WT) mice were obtained from Jackson Laboratories.

### Cancer Models

Tumour cells were orthotopically implanted into 6-8 week old mice. Before implantation, representative tumour cell lines were tested and found negative for common laboratory animal pathogens (Charles River). For experiments with rhabdomyosarcoma models, mice were orthotopically implanted with 1.5×10⁵ M3-9-M or M3-9-M^{OVA} cells in 50 μL of PBS into the right-behind leg gastrocnemius muscle using an insulin syringe. RMS tumour volume was calculated by subtracting the volume of the gastrocnemius muscle before tumour implantation from the volume after tumour implantation (length×width×height). 5×10⁵ B16.F10 melanoma tumour cells in 50 μL of PBS was implanted into the right flank of the mice using an insulin syringe. Melanoma tumour volume was determined by measuring the area of the implant site (length×width²×0.5). Tumour measurements were taken twice weekly using skin calipers, from implantation day to either experimental or humane endpoints.

### Microbiome Composition Analysis

Mice were gently restrained and fecal samples were collected directly into microfuge tubes. Fecal samples were stored at −80° C. for later use. Using the DNeasy PowerSoil Pro Kit (Qiagen™), microbial DNA from the fecal samples were extracted and purified. DNA concentration of all samples was adjusted to 25 ng/μL and sent to the Centre for Health Genomics and Informatics (CHGI; University of Calgary) for sequencing. At CHGI, the following was conducted: 16S V3-V4 rRNA gene amplicon library preparation; Kapa qPCR library quantification assay (Roche); and 600 Cycle MiSeq v3 sequencing (Illumina). The sequencing files were processed using the DADA2 R package (Nat. Methods 13, 581 (2016)) and amplicon sequence variants (ASVs) were identified. Using SILVA53 version 138 reference database maintained by DADA2, taxonomy to the ASVs was assigned. Bacterial abundance and richness, distance, and ordination was determined using the phyloseq R package (PLoS One 8, (2013)). A permutational analysis of variance (PERMANOVA) was performed using “pairwise.adonis” function (R package version 0.4. (2020)) with 999 permutations and Bonferroni adjustment for multivariate analysis.

### UHPLC-MS Untargeted Metabolomics

Snap frozen fecal, serum, and tumour samples were stored at −80° C. after collection. On the day of metabolite extraction, 20 mg of fecal or tumour mass or 20 μL of serum samples were thawed on ice followed by the addition of ice cold 50% methanol for extraction (1:50 dilution). Fecal and tumour samples were homogenized by bead beating, incubated on ice for 30 minutes, and centrifuged at 21000×g at 4° C. for 10 minutes. Half of the supernatants were collected and centrifuged again. Finally, 250 μL supernatant was collected from each sample for mass spectrometry analysis at the Calgary Metabolomics Research Facility (CMRF; University of Calgary), using ultra-high performance liquid chromatography mass spectrometry (UHPLC-MS) on a Q Exactive™ HF Mass Spectrometer (Thermo Scientific) in negative ion full scan mode (50-750m/z) at 240,000 resolution. To separate metabolites via UHPLC, a binary solvent mixture of 20 mM ammonium formate at pH 3.0 in LC-MS grade water (Solvent A) and 0.1% formic acid (% v/v) in LC-MS grade acetonitrile (Solvent B) was used in conjunction with a Syncronis™ column (Thermo Fisher Scientific). A flow rate of 600 μL/min was used to run the samples using the following gradient: 0-2 minutes, 100% B; 2-7 minutes, 100-80% B; 7-10 minutes, 80-5% B; 10-12 minutes, 5% B; 12-13 minutes, 5-100% B; 13-15 minutes, 100% B. For all runs the sample injection volume was 2 μL. XCMS and MAVEN software packages (Anal. Chem. 84, 5035 (2012); Curr. Protoc. Bioinformatics 14, (2012)) was used for primary analysis and identified metabolites by matching m/z signals and retention times to commercial standards. The data was further analyzed with MetaboAnalyst v.5.0 tool for statistical significance tests (Nat. Protoc. 17, 1735-1761 (2022)).

Identifying Specific Metabolite-Producing Bacteria within the Same Biospecimen

Fecal samples were collected from six IMDM-CR and six IMDM-JAX female C57BL/6 mice. Each sample was divided into two parts, with one part for UHPLC-MS untargeted metabolomics assay at CMRF and the other for 16S amplicon sequencing at CHGI. After filtering the initial metagenomic and metabolomic data, the MelonnPan R package (Nat. Commun. 10, 1-11 (2019)) was used to identify the bacteria that produce HPP metabolites. A table of paired sequence features and microbial community metabolite abundances were used as input into the “melonnnpan.train” function, which allowed for the unbiased identification of specific bacteria producing metabolites within the same biospecimen.

### Antibiotics-Mediated Dysbiosis

A broad-spectrum antibiotic cocktail consisting of ampicillin (1 mg/mL), neomycin (1 mg/mL), vancomycin (0.5 mg/mL), and metronidazole (1 mg/mL) was added to the drinking water of the mice. The concentration of metronidazole gradually increased, reaching 1 mg/mL on day 9. The antibiotics treatment was initiated two weeks before tumour inoculation and continued throughout the experiment, replacing the antibiotics-supplemented drinking water every 3-4 days. The well-being of the mice was monitored by measuring their body weights. To evaluate the effectiveness of the antibiotics, real-time polymerase chain reaction (RT-PCR) was performed on fecal microbial DNA using the universal oligonucleotide primers specific for conserved regions of the eubacterial 16S rRNA gene (forward primer, 5′¹³²⁰-CCATGAAGTCGGAATCGCTAG-¹³⁴¹3′; reverse primer, 5′¹⁴³¹-ACTCCCATGGTGTGACGG-¹⁴¹³3′). The Fecal microbial DNA was extracted using the DNeasy PowerSoil Pro Kit (Qiagen™) and a 20 μL PCR reaction mixture was prepared with iQ™ SYBR® Green supermix (Bio-Rad), 300 nM of each primer, 3 ng of DNAtemplate, and UltraPure™ DNase/RNase-Free Distilled Water (Invitrogen™). The Bio-Rad® CFX96™ RT-PCR system was used for thermal cycling, with a protocol of activation and denaturation of the polymerase at 95° C. for 5 minutes, followed by 35 cycles of denaturation at 95° C. for 15 seconds and annealing and extension at 60° C. for 1 minute.

### Flow Cytometry

Tumours were isolated from mice and single cell suspensions were made using a mouse tumour dissociation kit following the manufacturer's instructions (Miltenyi Biotec). The isolated tumours were minced into pieces ~2-4 mm in diameter using a sterile scalpel, homogenized the tumour samples in RPMI 1640 media and enzyme mix for 1 minute, and incubated the homogenates for 40 minutes with continuous gentle shaking at 37° C. The homogenates were then filtered through a 70 μm cell strainer followed by centrifuging the single cell suspensions. The red blood cells (RBC) were removed using RBC lysis buffer. A Percoll gradient based leukocyte separation was performed followed by live and dead cell staining using Zombie Aqua dye (BioLegend). Cell surface markers were stained using ant-CD4-FITC (clone RM4-4, BioLegend), anti-CD8-PE.Cy7 (clone 53-6.7, BioLegend), anti-CD3-BV421 (clone 145-2C11, BioLegend), anti-CD45-APC.Cy7 (clone 30-F11, BioLegend), anti-PD-1-FITC (clone 29F.1A12, BioLegend), anti-TIM3-PE (clone RMT3-23, BioLegend) and anti-LAG3-PerCP/Cy5.5 (clone C9B7W, BioLegend) for T lymphocytes. For OVA-specific CD8 T cell detection, H-2K(b) chicken ova 257-264 human B2M SIINFEKL Alexa 647-labeled tetramer was used. The cells were washed after staining with FACS buffer and quantified using the Attune NxT Flow Cytometer (Thermo Fisher Scientific).

### Mouse Treatment Regimens

Metabolite compounds were obtained from Sigma-Aldrich and prepared into metabolite stock solutions by dissolving them into UltraPure™ DNase/RNase-Free Distilled Water (Invitrogen™) with 1.0N NaOH (Sigma). The pH of the stock solutions was adjusted to 7.4-7.6 and determination of the endotoxin level of the solutions was completed through a limulus amebocyte lysate (LAL) test. First, the maximum tolerable dose and pharmacokinetics of the metabolite for animal experiments was established. To test the metabolite effects on anti-tumour immunity, 100 μL of the metabolite at 83 mg/kg bodyweight per mouse was administered intraperitoneally every 48 hours for seven doses. For immune checkpoint blockade (ICB) experiments, anti-PD-1 (clone RMP1-14, BioXcell) and anti-PD-L1 (clone 10F.9G2, BioXcell) antibodies were used. Three doses of 250 μg of either anti-PD-1 or anti-PD-L1 were administered intraperitoneally post tumour implantation on day 10, 13 and 16. To neutralize CD8 T cell in mice, anti-CD8 (clone 2.43; BioXcell) monoclonal antibody was used. 250 μg of anti-CD8 antibody in PBS per mouse was injected intraperitoneally 7 and 1 day before tumour implantation, followed by administering 100 μg of anti-CD8 in PBS into the mice on day 3, 7, and 10. The efficiency of T-cell depletion was monitored by flow cytometry using anti-CD8-PE.Cy7 antibody (clone 53-6.7, BioLegend).

### NF-κB and IRF Activity Reporter Assay

NF-κB and IRF induction assays were performed using mouse RAW-Dual™ (InvivoGen) and human THP1-Dua™ (InvivoGen) reporter cell lines. The cells were resuspended in freshly prepared test media at a concentration of 1.0×10⁶ cells/mL for RAW-Dual™ and 5.0×10⁵ cells/mL for THP1-Dual™. 180 μL of cells were seeded per well in a standard 96-well plate and optimized the concentration of several PRR agonists that trigger NF-κB and IRF. To test the metabolite effect on these pathways, 10 μL of the optimized receptor agonist, 10 μL of the 2 mM metabolite solution or vehicle was added to each well, making a final volume of 200 μL. To neutralize IL-1 receptor signalling, anti-IL-1a (clone 7D4, InvivoGen) and anti-IL-1β (clone 4H5, InvivoGen) antibodies were added and IgG1 isotype (clone T8E5, InvivoGen) was used as a control, as per manufacturer's recommendation. NF-κB activity was determined by measuring secreted embryonic alkaline phosphatase (SEAP) levels using QUANTI-Blue™ solution (InvivoGen). After aliquoting 20 μL of cell culture supernatant into 180 μL of QUANTI-Blue™, the conversion of color to blue was monitored and SEAP levels were determined using a SpectraMax i3 from Molecular Devices at 650 nm. To assess IRF induction, secreted lucia luciferase levels were measured in the culture medium using QUANTI-Luc™ solution (InvivoGen). 20 μL of cell culture supernatant was aliquoted into 50 μL of QUANTI-Luc™ solution and the luminescence reading was immediately recorded using the SpectraMax i3.

### Cell Viability and Cytotoxicity Assays

Cell viability and cytotoxicity assays were conducted using alamarBlue™ (Invitrogen™) and Roche's Cytotoxicity Detection KitPLUS (LDH). For cell viability, 10 μL of alamarBlue™ was mixed with 90 μL of cell culture per well in a 96-well plate, incubated at 37° C. with 5% CO₂, and fluorescence readings were taken using a SpectraMax i3. The culture medium with cells was used as the positive control and without cells as the negative control. For the LDH release assay, 46 μL of reaction mixture was mixed with 46 μL of cell culture supernatant into a 96-well plate, that was incubated in the dark for 20 minutes. Readings were taken with a SpectraMax i3. The positive control was the supernatant of the cells treated with the lysis solution, and the negative control was the culture medium without cells.

### Cytokine Assays

Cells were seeded in a 96-well plate at a density of 0.5-1.0×10⁶ cells/well and treated with PRR agonists in combination with or without metabolites. To measure cytokine levels, culture supernatant was collected at specific timepoints. To check cytokine levels of the tumour interstitial fluid, samples were prepared following the protocol, as mentioned by Kim et al (Nat. Commun. 8, (2017)). Cellular IL-1 levels were determined by incubating HEK-Blue™ IL-1β cells (Invitrogen™) with the collected culture supernatant and measuring the SEAP levels in the culture medium using QUANTI-Blue™ solution (Invitrogen™). Multiplexed quantification of cytokines, chemokines, and growth factors was also conducted by Eve Technologies Corp. (Calgary, Alberta) using the Luminex™ 200 system (Luminex, Austin, TX, USA).

Single Cell RNA Sequencing (scRNAseq)

M3-9-M^{OVA} RMS cells were inoculated orthotopically in male IMDM mice and a 3,2-HPP treatment regimen was initiated 1 day after tumour implantation. The final dose of treatment was administered 6 hours before tumour extraction to assess the impact on early gene expression in the context of overall survival experiments. After 14 days, tumours were extracted and minced into small pieces (~2-4 mm in diameter) using a sterile scalpel followed by the preparation of single cell suspensions using a tumour dissociation kit (Miltenyi Biotec). The density of viable cells was enriched using a dead cell removal kit (Miltenyi Biotec), and CD45+ tumour infiltrating immune cells isolated using mouse CD45 (TIL) mirobeads (Miltenyi Biotec). Flow cytometry was performed to verify the purity of the isolated CD45+ cell population (average of 95%) and the cells' health was confirmed via microscopy. Two samples were then pooled from each experimental group to enrich cell numbers for scRNA library construction. Chromium Single Cell 3′ GEM (Library and Gel Bead Kit v3) and the Chromium Controller platform were used to construct scRNA library, aiming for an estimated 5000 cells per library. The libraries were then sequenced using the NovaSeq™ 6000 platform (Illumina) at the CHGI (University of Calgary), with a single cartridge targeting 100,000 reads per cell.

scRNAseq Data Analysis

The raw sequencing files were processed using Cell Ranger 6.0.2 (10× Genomics). The raw base call (BCL) files were demultiplexed into FASTQ files using the “cellranger mkfastq” command (sample index: IMDM-CR=SI-GA-A3, IMDM-CR-HPP=SI-GA-B3 and IMDM-JAX=SI-GA-C3). The “cellranger count” pipeline was used to perform sequence alignment, filtering, barcode counting, UMI counting, and mapping to the 10× Genomics pre-built mouse reference genome (mm10, GENCODE vM23/Ensembl 98, 7 Jul. 2020). The “cellranger aggr” pipeline was used to aggregate the libraries in equal sequencing depth for analysis in Loupe Browser 5.1.0. The data was also analyzed using Seurat R package60-62, with results consistent with Loupe Browser analysis. Seurat was used to filter the data, integrate samples, normalize genes, reduce dimensions, and visualize the data. Cells with low or high feature count (<200 or >7500) and high mitochondrial gene frequency (>5%) were removed. All samples were integrated into a single Seurat object, the data scaled, linear and non-linear dimensional reduction performed, and cells clustered. The clusters were manually annotated using cell type specific expression markers (Nature 562, 367-372 (2018); Nat. Commun. 11, (2020); Nucleic Acids Res. 51, (2022); J. Immunother. Cancer 9, 1-18 (2021); J. Immunother. Cancer 10, 1-14 (2022); J. Exp. Med. 218, (2021)). Differentially expressed genes were identified using Wilcoxon rank sum test. Metascape (Nat. Commun. 10, (2019)) and Cytoscape (Genome Res. 13, (2003)) were used for pathway identification and protein network visualization.

### Thermal Profiling Using Intact Cells

24 mL of THP1-Dual™ cells (at a density of 1.5×106 cells/mL) were treated with 2 mM metabolite or vehicle control for 16 hours. The cells were then centrifuged at 340×g for 5 minutes at 4° C., resuspended in 20 mL of ice-cold PBS, and centrifuged again. The cells were resuspended in 1200 μL of PBS, divided into 10 aliquots of 100 μL in 0.2 mL PCR tubes and centrifuged at 325×g for 2 minutes at 4° C. 80 μL of the PBS supernatant was removed using a pipette and the tubes were gently tapped to resuspend the cells. Each of the metabolite and vehicle-treated cell tubes were then heated in parallel in a PCR machine for 3 minutes at temperatures ranging from 37° C. to 67° C. After incubating the cells at room temperature for 3 minutes, they were snap-froze in liquid nitrogen for 1 minute. The cells were briefly thawed in a 25° C. water bath, placed on ice, and resuspended using a pipette. The freeze-thaw step was repeated once and then 30 μL of PBS was added to the samples. The entire content was centrifuged at 100,000×g for 20 minutes at 4° C., the supernatant was removed into a fresh tube, and the protein concentration of the 37° C. sample was measured. The lysate was reduced using 10 mM dithiothreitol (DTT) at room temperature for 30 minutes and alkylated using 50 mM chloroacetamide (CAA) for 30 minutes in the dark. The volume of lysate that gave 200 μg of protein at the 37° C. temperature point was determined and removed into a fresh tube for all other temperature points.

### Protein Clean Up and Peptide Digestion

The proteins were cleaned up and the peptides digested following previously described methods (Science 346, (2014); Nat. Protoc. 10, 1567-1593 (2015)). The volume of lysate was made up from the 37° C. temperature point, equivalent to 200 μg, to 190 μL with 50 mM HEPES-NaOH pH 7.3 for each sample. The SP3 beads (Sera-Mag SpeedBeads, GE Healthcare, cat. no. 41552105050250 and 65152105050250) were washed 2 times with 180 μL of H₂O, resuspended in 10 μL of H₂O, and added to the sample along with 200 μL of 100% ethanol. The sample was then incubated on a thermomixer at room temperature for 10 minutes at 1000 rpm. The samples were washed 4 times with 180 μL of 80% ethanol and the beads resuspended in 200 μL of 50 mM HEPES-NaOH pH 7.3 with 4 μg of trypsin Lys C (Promega). The samples were incubated overnight at 37° C. on a shaking platform at 600 rpm. The next day, the digested peptides were transferred to a fresh Eppendorf™ tube and the concentration of peptides measured in the 37° C. temperature point.

### TMT Labelling

80 μL of 100% Acetonitrile was added to 0.8 mg of TMT reagents. The equivalent volume of 100 μg of peptide from the 37° C. temperature point was then selected and placed into a fresh eppendorf tube for each sample. 10 μL of TMT label was added to each sample and incubated at room temperature for 30 minutes. This process was repeated with the addition of 10 μL of TMT label. The TMT labelling was quenched with 10 μL of 1 M glycine. All 10 samples were then pooled and dried down using a speedy vac. 50% of the sample was desalted using Pierce Peptide Desalting Spin columns (cat. no. 89852) and resuspended in high pH mobile phase A (20 mM ammonium formate). The peptides were fractioned using High pH C18 Reverse phase HPLC with mobile phase B (100% ACN) with a 54 minute gradient (5%-80% B) at 0.2 mL/minute. The 54 fractions were concatenated into 18 fractions and dried down. Finally, the peptides were resuspended in 20 μL of 0.1% formic acid for mass spectrometry analysis. The NPARC R package (Mol. Cell. Proteomics 18, (2019)) was used for data analysis.

### Western Blotting

Cells were collected and lysates prepared using total lysis buffer (50 mM Tris-HCl, pH 8.0; 150 mM NaCl; 1% Triton X-100; 1% SDS). The supernatant was obtained by centrifuging at 10,000 RPM for 10 minutes at 4° C. using a benchtop PCR centrifuge. Protein quantification was performed using the Bio-Rad DC Protein Assay Kit (Bio-Rad, NC, USA). Proteins were separated using 12% SDS-PAGE gels and transferred to a PVDF membrane using the Bio-Rad Trans-turbo semi-dry transfer apparatus at 25 V/1 A for 1 hour. The membrane was blocked with Tris-buffered-saline-Tween-20™ (TBST) containing 5% skim milk for 30 minutes at room temperature. The membrane was then probed overnight at 4° C. with rabbit monoclonal recombinant anti-GSDMD antibody (ab210070, Abcam), anti-cleaved N-terminal GSDMD antibody (ab215203, Abcam) and mouse anti-actin monoclonal antibody (MAB1501, MilliporeSigma) for human FL-GSDMD, CL-GSDMD and actin. The next day, the membranes were washed with TBST three times and probed with goat anti-rabbit (1706515, Bio-Rad) or goat anti-mouse (1706516, Bio-Rad) horseradish peroxidase-conjugated IgG for 1 hour at room temperature. The secondary antibodies were diluted in TBST containing 5% (w/v) skim milk at 1:5000 and the membranes washed with TBST before detecting the proteins of interest using the Clarity™ Western ECL Substrate (Bio-Rad) on a Chemidoc-IT Imager (UVP, Upland, CA, USA).

### Sample Size Determination and Statistical Analysis

Sample sizes were predetermined for each experiment and pilot tests conducted to get a rough idea of the effect size. Using ClinCalc.com's power analysis tool (https://clincalc.com/stats/samplesize.aspx), the sample sizes were estimated for animal experiments. Random allocation of mice to experimental groups was ensured and mice were housed in multiple cages to eliminate cage effects. Prior experience and expert consultations also helped to determine sample sizes for certain techniques. All data points were included in the analysis and GraphPad Prism version 7.0 was used to analyze the tumour growth kinetics, animal survival, and in vitro experiment data. Statistical significance tests were performed for the TPP-TR, scRNAseq, and 16S amplicon sequencing data using R software packages. To determine the type of data distribution, the Shapiro-Wilk normality test or D'Agostino-Pearson omnibus normality test was performed. If the values formed a Gaussian or normal distribution, parametric statistical tests were performed, otherwise, non-parametric statistical tests were used. The confidence interval was set at 95% and the name of the statistical tests and P values in the corresponding figure or table legend reported.

While the present application has been described with reference to what are presently considered to be the preferred examples, it is to be understood that the application is not limited to the disclosed examples. To the contrary, the present application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE SPECIFICATION

Chen, D. S. & Mellman, I. Oncology Meets Immunology: The Cancer-Immunity Cycle. Immunity 39, 1-10 (2013).
Chen, D. S. & Mellman, I. Elements of cancer immunity and the cancer-immune set point. Nature 541, 321-330 (2017).
Osipov, A., Saung, M. T., Zheng, L. & Murphy, A. G. Small molecule immunomodulation: The tumor microenvironment and overcoming immune escape. Journal for Immuno Therapy of Cancer 7, (2019).
Binnewies, M. et al. Understanding the tumor immune microenvironment (TIME) for effective therapy. Nat. Med. 24, (2018).
Barth, H., Fischer, S., Moglich, A. & Fortsch, C. Clostridial C3 toxins target monocytes/macrophages and modulate their functions. Frontiers in Immunology 6, (2015).
Li, R. et al. Gut microbiota-stimulated cathepsin K secretion mediates TLR4-dependent M2 macrophage polarization and promotes tumor metastasis in colorectal cancer. Cell Death Differ. 26, (2019).
Gorjifard, S. & Goldszmid, R. S. Microbiota-myeloid cell crosstalk beyond the gut. J. Leukoc. Biol. 100, (2016).
Roy, S. & Trinchieri, G. Microbiota: A key orchestrator of cancer therapy. Nature Reviews Cancer 17, (2017).
Daillbre, R. et al. Trial watch: the gut microbiota as a tool to boost the clinical efficacy of anticancer immunotherapy. Oncoimmunology 9, 1-8 (2020).
Wang, Y. et al. Fecal microbiota transplantation for refractory immune checkpoint inhibitor-associated colitis. Nat. Med. (2018). doi:10.1038/s41591-018-0238-9
Davar, D. et al. Fecal microbiota transplant overcomes resistance to anti-PD-1 therapy in melanoma patients. Science 371, (2021).
Baruch, E. N. et al. Fecal microbiota transplant promotes response in immunotherapy-refractory melanoma patients. Science 371, (2021).
Uribe-Herranz, M. et al. Gut microbiota modulates adoptive cell therapy via CD8α dendritic cells and IL-12. JCI insight 3, e94952 (2018).
Sivan, A. et al. Commensal Bifidobacterium promotes antitumor immunity and facilitates anti-PD-L1 efficacy. Science 350, 1084-1089 (2015).
Simon, M. M. et al. A comparative phenotypic and genomic analysis of C57BL/6J and C57BL/6N mouse strains. Genome Biol. 14, 1-22 (2013).
Keane, T. M. et al. Mouse genomic variation and its effect on phenotypes and gene regulation. Nature 477, 289-294 (2011).
Mekada, K. et al. Genetic differences among C57BL/6 substrains. Exp. Anim. 58, 141-149 (2009).
Morgun, A. et al. Uncovering effects of antibiotics on the host and microbiota using transkingdom gene networks. Gut 64, 1732-1743 (2015).
Highfill, S. L. et al. Disruption of CXCR2-mediated MDSC tumor trafficking enhances anti-PD1 efficacy. Sci. Transl. Med. 6, (2014).
Mallick, H. et al. Predictive metabolomic profiling of microbial communities using amplicon or metagenomic sequences. Nat. Commun. 10, 1-11 (2019).
Lyubetskaya, A. et al. Assessment of spatial transcriptomics for oncology discovery. Cell Reports Methods 2, (2022).
Durham, N. M. et al. Oncolytic VSV Primes Differential Responses to Immuno-oncology Therapy. Mol. Ther. 25, (2017).
Childs, D. et al. Nonparametric Analysis of Thermal Proteome Profiles Reveals Novel Drug-binding Proteins. Mol. Cell. Proteomics 18, (2019).
Tsuchiya, K. et al. Gasdermin D mediates the maturation and release of IL-1a downstream of inflammasomes. Cell Rep. 34, (2021).
He, W. et al. Gasdermin D is an executor of pyroptosis and required for interleukin-1p secretion. Cell Res. 25, 1285-98 (2015).
Lv, T. et al. Targeting of GSDMD sensitizes HCC to anti-PD-1 by activating cGAS pathway and downregulating PD-L1 expression. J. Immunother. Cancer 10, (2022).
Jiang, Y. et al. Gasdermin D restricts anti-tumor immunity during PD-L1 checkpoint blockade. Cell Rep. 41, (2022).
Xi, G. et al. GSDMD is required for effector CD8+ T cell responses to lung cancer cells. Int. Immunopharmacol. 74, (2019).
Wang, W. J. et al. Downregulation of gasdermin D promotes gastric cancer proliferation by regulating cell cycle-related proteins. J. Dig. Dis. 19, (2018).
Yan, H. et al. Cisplatin induces pyroptosis via activation of meg3/nlrp3/caspase-1/gsdmd pathway in triple-negative breast cancer. Int. J. Biol. Sci. 17, (2021).
Ning, H. et al. Enhancer decommissioning by MLL4 ablation elicits dsRNA-interferon signaling and GSDMD-mediated pyroptosis to potentiate anti-tumor immunity. Nat. Commun. 13, 1-20 (2022).
Yang, Y. et al. Hydrogen inhibits endometrial cancer growth via a ROS/NLRP3/caspase-1/GSDMD-mediated pyroptotic pathway. BMC Cancer 20, (2020).
Peng, X. et al. Nuclear translocation of Gasdermin D sensitizes colorectal cancer to chemotherapy in a pyroptosis-independent manner. Oncogene 41, 5092-5106 (2022).
Harel, M. et al. Proteomics of Melanoma Response to Immunotherapy Reveals Mitochondrial Dependence. Cell 179, (2019).
Shi, J. et al. Cleavage of GSDMD by inflammatory caspases determines pyroptotic cell death. Nature 526, (2015).
Yang, J. et al. Mechanism of gasdermin D recognition by inflammatory caspases and their inhibition by a gasdermin D-derived peptide inhibitor. Proc. Natl. Acad. Sci. 115, (2018).
Mager, L. F. et al. Microbiome-derived inosine modulates response to checkpoint inhibitor immunotherapy. Science 369, 1481-1489 (2020).
Wang, T. et al. Inosine is an alternative carbon source for CD8+-T-cell function under glucose restriction. Nat. Metab. 2, (2020).
Wang, H. et al. The microbial metabolite trimethylamine N-oxide promotes antitumor immunity in triple-negative breast cancer. Cell Metab. 34, (2022).
Messaoudene, M. et al. A Natural Polyphenol Exerts Antitumor Activity and Circumvents Anti-PD-1 Resistance through Effects on the Gut Microbiota. Cancer Discov. 12, (2022).
Mirji, G. et al. The microbiome-derived metabolite TMAO drives immune activation and boosts responses to immune checkpoint blockade in pancreatic cancer. Sci. Immunol. 7, (2022).
Jneid, B. et al. Selective STING stimulation in dendritic cells primes antitumor T cell responses. Sci. Immunol. 8, (2023).
Lam, K. C. et al. Microbiota triggers STING-type I IFN-dependent monocyte reprogramming of the tumor microenvironment. Cell 184, (2021).
Steed, A. L. et al. The microbial metabolite desaminotyrosine protects from influenza through type I interferon. Science 357, 498-502 (2017).
Wei, Y. et al. The intestinal microbial metabolite desaminotyrosine is an anti-inflammatory molecule that modulates local and systemic immune homeostasis. FASEB J. 34, 16117-16128 (2020).
Zhao, H., Lazarenko, O. P. & Chen, J. R. Hippuric acid and 3-(3-hydroxyphenyl) propionic acid inhibit murine osteoclastogenesis through RANKL-RANK independent pathway. J. Cell. Physiol. 235, (2020).
Cheng, D. et al. Comparative Study on the Protective Effect of Chlorogenic Acid and 3-(3-Hydroxyphenyl) Propionic Acid against Cadmium-Induced Erythrocyte Cytotoxicity: In Vitro and in Vivo Evaluation. J. Agric. Food Chem. 69, (2021).
Evavold, C. L. et al. The Pore-Forming Protein Gasdermin D Regulates Interleukin-1 Secretion from Living Macrophages. Immunity 48, (2018).
Zhang, J. et al. Epithelial Gasdermin D shapes the host-microbial interface by driving mucus layer formation. Sci. Immunol. 7, (2022).
Han, C. et al. The AIM2 and NLRP3 inflammasomes trigger IL-1-mediated antitumor effects during radiation. Sci. Immunol. 6, (2021).
Ben-Sasson, S. Z. et al. IL-1 enhances expansion, effector function, tissue localization, and memory response of antigen-specific CD8 T cells. J. Exp. Med. 210, 491-502 (2013).
Callahan, B. J. et al. DADA2: High resolution sample inference from Illumina amplicon data. Nat. Methods 13, 581 (2016).
Quast, C. et al. The SILVA ribosomal RNA gene database project: improved data processing and web-based tools. Nucleic Acids Res. 41, (2013).
McMurdie, P. J. & Holmes, S. Phyloseq: An R Package for Reproducible Interactive Analysis and Graphics of Microbiome Census Data. PLoS One 8, (2013). Martinez Arbizu, P. Pairwise multilevel comparison using adonis. R package version 0.4. (2020). Available at: https://github.com/pmartinezarbizu/pairwiseAdonis.
Tautenhahn, R., Patti, G. J., Rinehart, D. & Siuzdak, G. XCMS Online: a web-based platform to process untargeted metabolomic data. Anal. Chem. 84, 5035 (2012).
Clasquin, M. F., Melamud, E. & Rabinowitz, J. D. LC-MS Data Processing with MAVEN: A Metabolomic Analysis and Visualization Engine. Curr. Protoc. Bioinformatics 14, (2012).
Pang, Z. et al. Using MetaboAnalyst 5.0 for LC-HRMS spectra processing, multi-omics integration and covariate adjustment of global metabolomics data. Nat. Protoc. 17, 1735-1761 (2022).
Kim, D. S. et al. Smac mimetics and oncolytic viruses synergize in driving anticancer T-cell responses through complementary mechanisms. Nat. Commun. 8, (2017).
Satija, R., Farrell, J. A., Gennert, D., Schier, A. F. & Regev, A. Spatial reconstruction of single-cell gene expression data. Nat. Biotechnol. 33, 495-502 (2015).
Butler, A., Hoffman, P., Smibert, P., Papalexi, E. & Satija, R. Integrating single-cell transcriptomic data across different conditions, technologies, and species. Nat. Biotechnol. 36, 411-420 (2018).
Stuart, T. et al. Comprehensive Integration of Single-Cell Data. Cell 177, 1888-1902 (2019).
Schaum, N. et al. Single-cell transcriptomics of 20 mouse organs creates a Tabula Muris. Nature 562, 367-372 (2018).
Yao, J. et al. Single-cell transcriptomic analysis in a mouse model deciphers cell transition states in the multistep development of esophageal cancer. Nat. Commun. 11, (2020).
Hu, C. et al. CellMarker 2.0: an updated database of manually curated cell markers in human/mouse and web tools based on scRNA-seq data. Nucleic Acids Res. 51, (2022).
Zhang, C. et al. Single-cell sequencing reveals antitumor characteristics of intratumoral immune cells in old mice. J. Immunother. Cancer 9, 1-18 (2021).
Costa, A. et al. Single-cell transcriptomics reveals shared immunosuppressive landscapes of mouse and human neuroblastoma. J. Immunother. Cancer 10, 1-14 (2022).
Veglia, F. et al. Analysis of classical neutrophils and polymorphonuclear myeloid-derived suppressor cells in cancer patients and tumor-bearing mice. J. Exp. Med. 218, (2021).
Zhou, Y. et al. Metascape provides a biologist-oriented resource for the analysis of systems-level datasets. Nat. Commun. 10, (2019).
Shannon, P. et al. Cytoscape: A Software Environment for Integrated Models of Biomolecular Interaction Networks. Genome Res. 13, (2003).
Savitski, M. M. et al. Tracking cancer drugs in living cells by thermal profiling of the proteome. Science 346, (2014).
Franken, H. et al. Thermal proteome profiling for unbiased identification of direct and indirect drug targets using multiplexed quantitative mass spectrometry. Nat. Protoc. 10, 1567-1593 (2015).

1.-22. (canceled)

23. A method of increasing efficacy of immunotherapy in a patient with cancer comprising administering to the patient an effective amount of one or more compounds of Formula (V), a pharmaceutically acceptable salt, solvate and/or ester thereof: 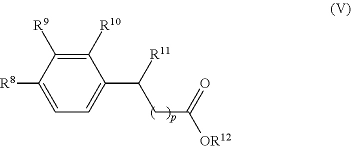
wherein:
R⁸, R⁹ and R¹⁰ are, independently OH or H, provided at least one of R⁸, R⁹ and R¹⁰ is OH, or one of R⁸, R⁹ and R¹⁰ is selected from halogen, NR¹³R¹⁴ and C₁₋₃ alkyl, and the other two of R⁸, R⁹ and R¹⁰ are H;
R¹³ and R¹⁴ are independently selected from H and C₁₋₃ alkyl;
R¹¹ is selected from H and =0;
R¹² is selected from H, C₁₋₄alkyl and succinimide; and p is 1-4.

24. The method of claim 23, wherein the one or more compounds are selected from the compounds of formula (I), (II), (III) or (IV), a pharmaceutically acceptable salt, solvate and/or ester thereof: 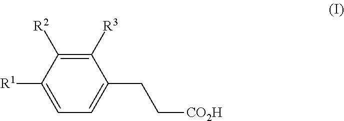
wherein: R¹, R² and R³ are, independently OH or H, provided at least one of R¹, R² and R³ is OH; 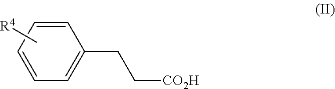
wherein: R⁴ is selected from halogen, NR⁵R⁶ and C₁₋₅ alkyl; and R⁵ and R⁶ are independently selected from H and C₁₋₃ alkyl; 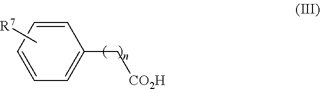
wherein: R⁷ is OH; and n is 3-5; and

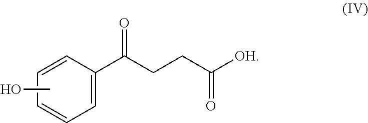

25. The method of claim 23, wherein the method further comprises pre-treating the patient with a broad-spectrum antibiotic, optionally at a dose that significantly reduces fecal bacteria without impacting body weight of the patient.

26. The method of claim 23, wherein the one of more compounds are selected from:
3-(4-fluorophenyl)propionic acid,
3-(4-chloroyphenyl)propionic acid,
3-(4-aminoyphenyl)propionic acid,
3-(4-methylphenyl)propionic acid,
3-(4-hydroxyphenyl)propionic acid,
4-(4-hydroxyphenyl)butanoic acid,
5-(4-hydroxyphenyl)pentanoic acid; and
a pharmaceutically acceptable salt thereof.

27. The method of claim 23, wherein the one or more compounds are selected from 3,2-hydroxyphenyl propionic acid, 3,3-hydroxyphenyl propionic acid and 3,4-hydroxyphenyl propionic acid, or a pharmaceutically acceptable salt thereof.

28. The method of claim 23, wherein the ester of the one or more compounds is selected from methyl 3-(4-hydroxyphenyl) propionate, ethyl 3-(4-hydroxyphenyl)propionate, and 3-(4-hydroxyphenyl)propionic acid N-hydroxysuccinimide ester.

29. The method of claim 23, wherein the immunotherapy is an immune checkpoint blockade agent.

30. The method of claim 29, wherein the immune checkpoint blockade agent is a PD-1 inhibitor optionally Pembrolizumab, Nivolumab, and/or Cemiplimab or a PD-L1 inhibitor including optionally Atezolizumab, Avelumab, and/or Durvalumab.

31. The method of claim 29, wherein the immune checkpoint blockage agent is a CTLA-4 inhibitor optionally Ipilimumab and/or Tremelimumab.

32. The method of claim 29, wherein the immune checkpoint blockage agent is a LAG-3 inhibitor optionally Relatlimab and/or Opdualag.

33. The method of claim 23, wherein the patient, prior to said method is non-responsive or minimally responsive to the immunotherapy or predicted to be non-responsive or minimally responsive to the immunotherapy.

34. The method of claim 33, wherein the method further comprises pre-treating the patient with a broad-spectrum antibiotic, optionally at a dose that significantly reduces fecal bacteria without impacting body weight of the patient.

35. The method of claim 33, wherein the one or more compounds are selected from the compounds of formula (I), (II), (III) or (IV), a pharmaceutically acceptable salt, solvate and/or ester thereof: 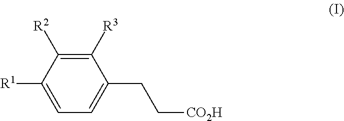
wherein: R¹, R² and R³ are, independently OH or H, provided at least one of R¹, R² and R³ is OH; 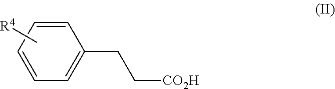
wherein: R⁴ is selected from halogen, NR⁵R⁶ and C₁₋₅ alkyl; and R⁵ and R⁶ are independently selected from H and C₁₋₃ alkyl; 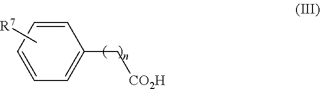
wherein: R⁷ is OH; and n is 3-5; and

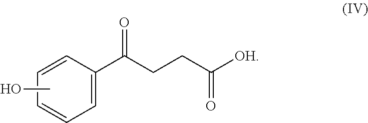

36. The method of claim 33, wherein the one of more compounds are selected from:
3-(4-fluorophenyl)propionic acid,
3-(4-chloroyphenyl)propionic acid,
3-(4-aminoyphenyl)propionic acid,
3-(4-methylphenyl)propionic acid,
3-(4-hydroxyphenyl)propionic acid,
4-(4-hydroxyphenyl)butanoic acid,
5-(4-hydroxyphenyl)pentanoic acid; and
a pharmaceutically acceptable salt thereof.

37. The method of claim 33, wherein the one or more compounds are selected from 3,2-hydroxyphenyl propionic acid, 3,3-hydroxyphenyl propionic acid and 3,4-hydroxyphenyl propionic acid, or a pharmaceutically acceptable salt thereof.

38. The method of claim 33, wherein is the esters of the one or more compounds are selected from methyl 3-(4-hydroxyphenyl) propionate, ethyl 3-(4-hydroxyphenyl)propionate and 3-(4-hydroxyphenyl)propionic acid N-hydroxysuccinimide ester.

39. The method of claim 33, wherein the immunotherapy is an immune checkpoint blockade agent.

40. The method of claim 39, wherein the immune checkpoint blockade agent is a PD-1 inhibitor optionally Pembrolizumab, Nivolumab, and/or Cemiplimab or a PD-L1 inhibitor optionally Atezolizumab, Avelumab, and/or Durvalumab.

41. The method of claim 39, wherein the immune checkpoint blockage agent is a CTLA-4 inhibitor including Ipilimumab and/or Tremelimumab.

42. The method of claim 39, wherein the immune checkpoint blockage agent is a LAG-3 inhibitor including Relatlimab and/or Opdualag.

43. A method of increasing anti-tumour immunity, comprising administering to a patient in need thereof an effective amount of one or more compounds Formula (V), a pharmaceutically acceptable salt, a solvate and/or ester thereof: 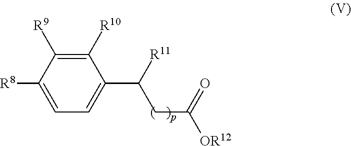
wherein:
R⁸, R⁹ and R¹⁰ are, independently OH or H, provided at least one of R⁸, R⁹ and R¹⁰ is OH, or one of R⁸, R⁹ and R¹⁰ is selected from halogen, NR¹³R¹⁴ and C₁₋₃ alkyl, and the other two of R⁸, R⁹ and R¹⁰ are H;
R¹³ and R¹⁴ are independently selected from H and C₁₋₃ alkyl;
R¹¹ is selected from H and ═O;
R¹² is selected from H, Ci.₄alkyl and succinimide; and p is 1-4.

44. The method of claim 43, wherein the one or more compounds are selected from the compounds of formula (I), (II), (III) or (IV), a pharmaceutically acceptable salt, solvate and/or ester thereof: 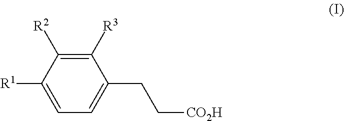
wherein: R¹, R² and R³ are, independently OH or H, provided at least one of R¹, R² and R³ is OH; 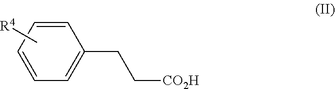
wherein: R⁴ is selected from halogen, NR⁵R⁶ and C₁₋₅ alkyl; and R⁵ and R⁶ are independently selected from H and C₁₋₃ alkyl; 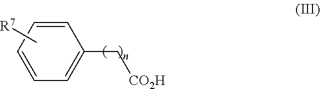
wherein: R⁷ is OH; and n is 3-5; and

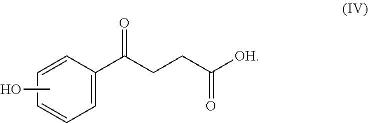

45. The method of claim 43, wherein the method further comprises pre-treating the patient with a broad-spectrum antibiotic, optionally at a dose that significantly reduces fecal bacteria without impacting body weight of the patient.

46. The method of claim 43, wherein the compounds are selected from:
3-(4-fluorophenyl)propionic acid,
3-(4-chloroyphenyl)propionic acid,
3-(4-aminoyphenyl)propionic acid,
3-(4-methylphenyl)propionic acid,
3-(4-hydroxyphenyl)propionic acid,
4-(4-hydroxyphenyl)butanoic acid,
5-(4-hydroxyphenyl)pentanoic acid; and
a pharmaceutically acceptable salt thereof.

47. The method of claim 43, wherein the one or more compounds are selected from 3,2-hydroxyphenyl propionic acid, 3,3-hydroxyphenyl propionic acid and 3,4-hydroxyphenyl propionic acid, or a pharmaceutically acceptable salt thereof.

48. The method of claim 43, wherein the ester of the one or more compounds is selected from methyl 3-(4-hydroxyphenyl) propionate, ethyl 3-(4-hydroxyphenyl)propionate, and 3-(4-hydroxyphenyl)propionic acid N-hydroxysuccinimide ester.

49. The method of claim 33, wherein the patient is predicted to be non-responsive or minimally responsive to the immunotherapy by determining fecal metabolomic profile of the patient, wherein if the fecal metabolomic profile is low in hydroxyphenyl propanoate (HPP) metabolites compared to a fecal metabolomic profile of known responders to the immunotherapy, the patient is predicted to be non-responsive or minimally responsive to the immunotherapy.
